(19) United States
(12) Patent Application Publication
(10) Pub. No.: US 2025/0325207 A1
(43) Pub. Date: Oct. 23, 2025

(54) PERSONALIZED TRAINING SYSTEM TO IMPROVE RECIPROCAL EYE ENGAGEMENT AND FACIAL EMOTIONAL SKILLS FOR NEURODIVERGENT INDIVIDUALS

(71) Applicant: Claire Xu, Cupertino, CA (US)

(72) Inventor: Claire Xu, Cupertino, CA (US)

(21) Appl. No.: 18/915,636

(22) Filed: Oct. 15, 2024

Related U.S. Application Data
(60) Provisional application No. 63/637,488, filed on Apr. 23, 2024.

(51) Int. Cl. A61B 5/16; A61B 5/00; G16H 50/20

(52) U.S. Cl. CPC A61B 5/163; A61B 5/165; A61B 5/4836; A61B 5/7267; G16H 50/20

(57) ABSTRACT

Provided herein are methods, devices, and systems for method for training a subject diagnosed with a socio-emotional skills deficit using engagement training to enhance dyadic behavior, comprising: selecting a first visual cue that will engage a visual attention of the subject; exposing the subject to the first visual cue for one or more visual cue cycles; recording at least one of: a focus location on screen, one or more timestamps, tracking one or more directions of gaze, one or more blinks, or a reciprocal gaze; processing the recorded tracking one or more directions of gaze of the one or more eyes to determine a dwell time of the one or more directions of gaze and repeating the step of exposing the subject, wherein an increase of the dwell time, reciprocal gaze, or both, are indicative that the subject diagnosed the socio-emotional skills deficit has increased focus and attention.

### CROSS-REFERENCE TO RELATED APPLICATIONS

This application claims priority to U.S. Provisional Application Ser. No. 63/637,488, filed Apr. 23, 2024, the entire contents of which are incorporated herein by reference.

### TECHNICAL FIELD OF THE INVENTION

The present invention relates in general to the field of personalized training system to improve reciprocal eye engagement and facial emotional skills for neurodivergent individuals, and more particularly, the system uses computer vision, multimodal models and digital twins for such training, and is designed based on the relevant neuroscience theories (Amygdala Theory, Mirror Neuron System, Relevance Detector Theory, Theory of Mind).

### STATEMENT OF FEDERALLY FUNDED RESEARCH

None.

### INCORPORATION-BY-REFERENCE OF MATERIALS FILED ON COMPACT DISC

None.

### BACKGROUND OF THE INVENTION

Without limiting the scope of the invention, its background is described in connection with personalized training system for neurodivergent individuals.

Social cognition, especially emotional understanding, has become the centerpiece of many trending research focuses: Autism Spectrum Disorder (ASD), Depression and anxiety, and schizophrenia. One of the primary symptoms of ASD is the lack of emotional recognition and expression. Increasing evidence suggests that these difficulties are due to a high rate of alexithymia among autistic people. According to senior investigator Geoff Bird, professor of cognitive neuroscience at the University of Oxford in the United Kingdom, about 50 percent of autistic people have alexithymia, compared with 5 percent of non-autistic people [1]. Depression and anxiety are also associated with alexithymia with a prevalence rate of 26.9% in adults with depression [2]. In addition, significant alterations in emotion processing, with a tendency to appraise neutral stimuli as negative, leads patient to preferentially respond to negative stimuli [3]. Alexithymia is a common, but less recognized affective deficit in patients with schizophrenia with a prevalence rate ranging from 30 to 46%.

For example, alexithymia is a common co-occurring condition for many neurodivergent conditions. The word alexithymia was derived from Greek (a=lack, lexis=word, thymos=emotion) to describe deficiencies in emotional functioning [5]. Alexithymia is not regarded as a disorder in the Diagnostic and Statistical Manual [4]. Alexithymia refers to people who have trouble identifying and describing emotions and who tend to minimize emotional experience and focus attention externally [6].

The challenges in the current therapy training are: The training content is not highly personalized. During training, emotion expressions are usually demonstrated on neurotypical people's faces, lacking analysis of neurodivergent individual's own facial expressions. There is a gap between their intention and others' perception. Also, the training material is not personalized. Emotion learning is not an innate process for neurodivergent population, currently it lacks precise explanation of the expected expressions. The training feedback and improvements are tracked by imprecise human observation. No timely feedback on subtle improvement. This population requires a much longer learning journey than the neurotypical population and hence it's critical to encourage them by reporting each subtle improvement. Finally, current therapy is not scalable, it's offered in 1:1 or small group setting which is resource intensive, costly: $120-150 per hour.

The challenges in the current Artificial Intelligence (AI)/Machine Learning (ML) models are: AI models output discrete emotion categories, but the actual emotion expressions are on the spectrum (continuous stages); and existing facial emotion models are not trained for neurodivergent populations and do not represent some of the characteristics, e.g. asymmetry, slide glancing. However, existing methods do not take into account the differences in the data streams, the ability of the subject to even engage with the images, and fails to provide a personalized method of knowing the reaction, gaze, dwell time, etc., of the specific subject or be able to adapt to the progression of the user during the course of treatment. Existing AI/ML systems are unable to do with by processing real-time images of the subject, and/or provide the specificity needed for individual users. What is needed are novel AI/ML methods for processing video images of an individual, personalizing the data from the same, and then applying a treatment, and tracking progress of the same.

What is needed are novel personalized methods and systems to provide precise real-time feedback in the training of reciprocal eye engagement and facial emotional skills for neurodivergent individuals.

### SUMMARY OF THE INVENTION

As embodied and broadly described herein, an aspect of the present disclosure relates to a method for training a subject diagnosed with a socio-emotional skills deficit using engagement training to enhance dyadic behavior, comprising: selecting a first visual cue that will engage a visual attention of the subject; exposing the subject to the first visual cue for one or more visual cue cycles comprising (1) a first time period with exposure of the subject to the visual cue, (2) a second time period that is a resting period in which the subject is not exposed to the first visual cue or the subject closes their eyes, and (3) a third time period in which the subject is not exposed to visual cues; recording at least one of: a focus location on screen, one or more timestamps, tracking one or more directions of gaze, one or more blinks, or a reciprocal gaze with the first visual cue for one or more eyes of the subject in conjunction with exposure to the one or more visual cue cycles; processing the recorded tracking one or more directions of gaze of the one or more eyes to determine a dwell time of the one or more directions of gaze toward the visual cue during the first time period of the one or more visual cue cycles; and administering a treatment based on the recorded tracking by administering a treatment based on the recorded tracking by the step of exposing the subject to the one or more visual cue cycles one or more times a day, until the dwell time of the eyes, reciprocal gaze, or both, exceeds a pre-set period of time, wherein an increase of the dwell time, reciprocal gaze, or both, are indicative that the subject diagnosed with the socio-emotional skills deficit has increased focus and attention. In one aspect, the method further comprises, once the dwell time, reciprocal gaze, or both of the eye meets or exceeds a preset period of time, then: selecting a second visual cue that will engage the visual attention of the subject; and administering a treatment based on the recorded tracking by repeating the steps of exposing, recording, and determining the gaze tracking of the subject to the second visual cue until the dwell time of the eyes exceeds a second pre-set period of time. In another aspect, the first visual cue is a trainer or video of a trainer on computer screen. In another aspect, the first time period is selected from 1, 1.5, 2, 2.5, 3, 3.5, 4, 4.5, 5, 5.5, 6, 6.5, 7, 7.5, 8, 8.5, 9, 9.5, 10, 15, 20, 25, or 30 seconds. In another aspect, the second time period is selected from 1, 2, 3, 4, 5, 6, 7, 8, 9, 10, 15, 20, 25, 30, 45, or 60 seconds. In another aspect, the third time period is selected from 1, 2, 3, 4, 5, 6, 7, 8, 9, 10, 15, 20, 25, 30, 45, 60 minutes, 2, 3, 4, 5, 6, 7, 8, 9, 10, 12, 14, 16, 18, 20, 24 hours, 2, 3, 4, 5, 6, 7 days. In another aspect, the first visual cue is selected from a face contour with a region covering the eyes, a cartoon of a face, a cartoon of an animal face, one or more neutral faces with one or more different age ranges, one or more different ethnicities, the face of the subject or a family member of the subject; or a trainer. In another aspect, the first visual cue can be one or more images, pre-recorded videos and live video feed from webcam, attached phone, or camera. In another aspect, a processor is programmed with a machine learning algorithm that calculates the position of one or more landmarks on a face selected from a position of the eye(s), eye contour, eyelid, pupil, a focus location on screen, one or more directions of gaze, blinks, dwell time, reciprocal eye engagement with a target in a training visual cue on a computer screen in conjunction with one or more timestamps during the exposure to the one or more visuals cue for one or more eyes of a subject; and wherein the one or more directions of gaze are selected from: (1) right, left, center; (2) up or down; or (3) combinations of (1) and (2); wherein the one or more landmarks on the face is calculated using 1, 2, 3, 4, 5, 6, 7, 8, 9, 10, 15, 20, 25, 30, 45, 60, 68, 70, 75, 80, 90, 100, 200, 300, 400, 500, 900, or 1000 facial landmarks. In another aspect, the one or more visual cue are repeated for 1, 2, 4, 5, 6, 7 days, 1, 2, 3, 4, 5, 6, 7, 8, 9, 10, 11, or 12 weeks, or 1, 2, 3, 4, 5, 6, 7, 8, 9, 10, 11, or 12 months until the dwell time, the reciprocal gaze, or both, meets or exceeds the pre-set period of time. In another aspect, the pre-set period of time is 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5 seconds. In another aspect, the first, the second and one or more additional visual cue(s) is/are, in order: (1) a cartoon of a face or a cartoon of an animal face; (2) one or more neutral faces with one or more age ranges or ethnicities; (3) one or more neutral faces with one or more different age ranges or ethnicities; (4) the face of the subject or a family member of the subject, and (5) a trainer. In another aspect, the method further comprises using a machine learning algorithm to continuously modify: a length of exposure to one or more visual cues, to increase the dwell time, reciprocal gaze time, or both, of the one or more eyes of the subject on the visual cue. In another aspect, the alexithymia is related to the subject having at least one of: autism, neurotypical, depression, anxiety, schizophrenia, or a deficit in recognizing or describing emotions. In another aspect, the training is provided on a computer, laptop, tablet, phone, or other electronic or handheld device. In another aspect, the socio-emotional skills deficit has at least one of: alexithymia, autism, depression, anxiety, or schizophrenia. In another aspect, the method is a self paced multi-level training based on personal preferences (geometry preference, animal preference, hyperlexia). In another aspect, the method uses digital twin training partners to represent self, family members and therapists to at least one of: (1) reduce stimuli, (2) bridge a gap between self intention and others' perception, and (3) practice the mirror neuron system. The method of claim 1, wherein the method uses digital twin training partners to represent self, family members and therapists to at least one of: (1) reduce stimuli, (2) bridge a gap between self-intention and others' perception, and (3) practice the mirror neuron system. A digital twin is created to resemble the real person's facial characteristics and emotion expressions. Trainees can watch the digital twins' videos for the eye engagement training and mimic digital twins' facial expressions for the emotion training. Also, the digital twins can mirror trainee's facial expressions. In another aspect, the method further comprises building a dataset that represents a neurodivergent individuals' facial features, a computer vision-based machine learning co-pilot feedback to augment neurological dysfunction, and one or more facial emotion expressions using selected facial features.

As embodied and broadly described herein, an aspect of the present disclosure relates to a method for facial emotional analysis of a subject, comprising: recording or obtaining one or more images or video of a face of the subject; detecting one or more facial feature in the one or more images or video of the face of the subject at least one of: a contour of the face, a focus location on a screen of one or more eyes, the irises of the one or more eyes, tracking one or more directions of gaze of the one or more eyes, a position of the one or more eyes, one or more blinks, a reciprocal gaze with a first visual cue for one or more eyes, a position of the mouth, a shape of the mouth, one or more eyebrows, a position of the one or more eyebrows, a share of the one or more eyebrows, of the subject in conjunction with exposure to the one or more visual cue cycles; selecting a first visual cue that will engage a visual attention of the subject; using a machine learning algorithm to detect one or more emotions selected from one or more selected from happy, sad, calm, triumph, aesthetic appreciations, relied, pride, admiration, adoration, contentment, satisfaction, love, excitement, interest, awe, amusement, joy, or ecstasy, in the facial features to generate a facial emotion analysis.

As embodied and broadly described herein, an aspect of the present disclosure relates to a device for training a subject diagnosed a socio-emotional skills deficit using engagement training to enhance dyadic behavior, comprising: a memory; a control circuitry functionally coupled to the memory, configured to: deliver to the subject a first visual cue that will engage a visual attention of the subject for one or more visual cue cycles comprising: (1) a first time period with exposure of the subject to the visual cue; (2) a second time period that is a resting period in which the subject is not exposed to the first visual cue or the subject closes their eyes; and (3) a third time period in which the subject is not exposed to visual cues; one or more cameras capable or capturing an image/video of the subject in conjunction with one or more timestamps during the one or more visual cue cycles; a processor that is programmed with a machine learning algorithm that processes the one or more captured image/video for the subject to calculate a position of one or more facial landmarks selected from at least one of: a position of the eyes, eye contour, eyelid, pupil, a focus location on a screen, one or more directions of gaze, one or more blinks, dwell time, or reciprocal eye engagement with target eyes in a training visual cue on a computer screen in conjunction with the one or more timestamps during exposure to the first visual cue for one or more eyes of the subject during the first time period of the one or more visual cue cycles; and wherein the subject is exposed to the one or more visual cue cycles one or more times a day, until the dwell time of the eyes, reciprocal gaze, or both, exceeds a pre-set period of time, and wherein an increase of the dwell time, reciprocal gaze, or both, is indicative that the subject with the socio-emotional skills deficit has increased focus and attention. In another aspect, the device further comprises a user interface, wherein the user interface is used to select from visual cues stored in said memory. In another aspect, the control circuitry is configured to determine when the dwell time, reciprocal gaze, or both, of the subject meets or exceeds the pre-set period of time. In another aspect, a second visual cue that will engage the visual attention of the subject and administering a treatment based on the recorded tracking by repeating the steps of exposing, recording, and determining a period of the gaze of the subject to the second visual cue until the dwell time, reciprocal gaze, or both, of the eyes exceeds a second pre-set period of time. In another aspect, the first time period is selected from 1, 1.5, 2, 2.5, 3, 3.5, 4, 4.5, 5, 5.5, 6, 6.5, 7, 7.5, 8, 8.5, 9, 9.5, 10, 15, 20, 25, or 30 seconds. In another aspect, the second time period is selected from 1, 2, 3, 4, 5, 6, 7, 8, 9, 10, 15, 20, 25, 30, 45, or 60 seconds. In another aspect, the third time period is selected from 1, 2, 3, 4, 5, 6, 7, 8, 9, 10, 15, 20, 25, 30, 45, 60, minutes, 2, 3, 4, 5, 6, 7, 8, 9, 10, 12, 14, 16, 18, 20, 24 hours, 2, 3, 4, 5, 6, 7 days. In another aspect, the first visual cue is selected from a face contour with a region covering the eyes, a cartoon of a face, a cartoon of an animal face, one or more neutral faces with one or more different age ranges, one or more different ethnicities, the face of the subject or a family member of the subject, or a trainer. In another aspect, the visual cue are selected from at least one of: one or more images, one or more pre-recorded videos, one or more live video feeds, from a webcam, a phone, or a camera. In another aspect, a processor is programmed with a machine learning algorithm that calculates the position of one or more landmarks on a face selected from a position of the eye(s), eye contour, eyelid, pupil, a focus location on screen, one or more directions of gaze, blinks, dwell time, reciprocal eye engagement with a target in a training visual cue on a computer screen in conjunction with one or more timestamps during the exposure to the one or more visuals cue for one or more eyes of a subject; and wherein the one or more directions of gaze are selected from: (1) right, left, center; (2) up or down; or (3) combinations of (1) and (2); wherein the one or more landmarks on the face is calculated using 1, 2, 3, 4, 5, 6, 7, 8, 9, 10, 15, 20, 25, 30, 45, 60, 68, 70, 75, 80, 90, 100, 200, 300, 400, 500, 900, or 1000 facial landmarks. In another aspect, the one or more visual cue cycles are repeated for 1, 2, 4, 5, 6, 7 days, 1, 2, 3, 4, 5, 6, 7, 8, 9, 10, 11, or 12 weeks, or 1, 2, 3, 4, 5, 6, 7, 8, 9, 10, 11, or 12 months until the dwell time, the reciprocal gaze, or both, meets or exceeds the pre-set period of time. In another aspect, the pre-set period of time is 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, or 3.0, 3.1, 3.2, 3.3, 3.4, 3.5 seconds. In another aspect, the first, the second, and one or more additional visual cue(s) is/are, in order: (1) a cartoon of a face or a cartoon of an animal face; (2) one or more neutral faces with one or more different age ranges, (3) one or more different ethnicities; (4) the face of the subject or a family member of the subject, and (5) a trainer. In another aspect, the device further comprises programming the processor with a machine learning algorithm to continuously modify: a length of exposure to one or more visual to increase the dwell time, reciprocal gaze time, or both, of the eyes of the subject on the visual cue. In another aspect, the position of the eyes on a device screen is computed using a machine learning algorithm based on one or more facial landmarks of the subject and real time changes when tracking a predefined moving visual cue on a device screen. In another aspect, the device further comprises displaying at least one of: one or more dwell times during and between one or more visual cue cycles; average dwell times across one or more visual cue cycles; average dwell times over various days, weeks, or months; or for a new visual cue one or more dwell times during and between one or more visual cue cycles; average dwell times across one or more visual cue cycles; average dwell times over various days, weeks, or months. In another aspect, the device further comprises displaying at least one of: one or more reciprocal gaze times during and between one or more visual cue cycles; average reciprocal gaze times across one or more visual cue cycles; average reciprocal gaze times over various days, weeks, or months; or for a subsequent visual cue one or more reciprocal gaze times during and between one or more visual cue cycles; average reciprocal gaze times across one or more visual cue cycles; average reciprocal gaze times over various days, weeks, or months. In another aspect, the alexithymia is related to the subject having one or multiple conditions selected from: autism, neurotypical, depression, anxiety, or schizophrenia or a subject with a deficit in recognizing or describing emotions. In another aspect, the training is provided on a computer, laptop, tablet, phone, or other electronic or handheld device. In another aspect, the socio-emotional skills deficit has at least one of: alexithymia, autism, depression, anxiety, or schizophrenia.

As embodied and broadly described herein, an aspect of the present disclosure relates to a device for generating a facial emotion analysis, comprising: recording or obtaining one or more images or video of a face of the subject; detecting one or more facial feature in the one or more images or video of the face of the subject at least one of: a contour of the face, a focus location on a screen of one or more eyes, the irises of the one or more eyes, tracking one or more directions of gaze of the one or more eyes, a position of the one or more eyes, one or more blinks, a reciprocal gaze with a first visual cue for one or more eyes, a position of the mouth, a shape of the mouth, one or more eyebrows, a position of the one or more eyebrows, a share of the one or more eyebrows, of the subject in conjunction with exposure to the one or more visual cue cycles; selecting a first visual cue that will engage a visual attention of the subject; using a machine learning algorithm to detect one or more emotions selected from triumph, aesthetic appreciations, relied, pride, admiration, adoration, contentment, satisfaction, love, excitement, interest, awe, amusement, joy, or ecstasy, in the facial features to generate a facial emotion analysis.

As embodied and broadly described herein, an aspect of the present disclosure relates to a computer or electronic system, comprising: one or more processors; and one or more hardware storage devices having stored thereon computer-executable instructions that, when executed by the one or more processors, configure the computer system to perform at least the following: selecting a first visual cue that will engage a visual attention of the subject; exposing the subject to the visual cue for one or more visual cue cycles comprising (1) a first time period with exposure of the subject to the visual cue, (2) a second time period that is a resting period in which the subject is not exposed to the first visual cue or the subject closes their eyes, and (3) a third time period in which the subject is not exposed to visual cues; recording a focus location on a screen, one or more timestamps, one or more directions of gaze, one or more blinks, or a reciprocal gaze with a trainer or a video of a trainer on computer screen for the one or more eyes of the subject to track a gaze in conjunction with exposure to the visual cue; processing the recorded gaze tracking of the one or more eyes to determine a dwell time of the gaze toward the visual cue during the first time period of the one or more visual cue cycles; and administering a treatment based on the recorded tracking by repeating the step of exposing the subject to the one or more visual cue cycles one or more times a day, until the dwell time of the eyes exceeds a pre-set period of time, wherein an increase of the dwell time, reciprocal gaze, or both, are indicative that the subject diagnosed with alexithymia or a subject with deficit in socio-emotional skills has increased focus and attention. In another aspect, the computer or electronic system further comprises, once the dwell time, reciprocal gaze, or both, of the eye meets or exceeds the pre-set period of time, then: selecting a second visual cue that will engage the visual attention of the subject; and administering a treatment based on the recorded tracking by repeating the steps of exposing, recording, and determining a period of the gaze of the subject to the second visual cue until the dwell time of the eyes exceeds a second pre-set period of time. In another aspect, the first time period is selected from 1, 1.5, 2, 2.5, 3, 3.5, 4, 4.5, 5, 5.5, 6, 6.5, 7, 7.5, 8, 8.5, 9, 9.5, 10, 15, 20, 25, or 30 seconds. In another aspect, the second time period is selected from 1, 2, 3, 4, 5, 6, 7, 8, 9, 10, 15, 20, 25, 30, 45, or 60 seconds. In another aspect, the third time period is selected from 1, 2, 3, 4, 5, 6, 7, 8, 9, 10, 15, 20, 25, 30, 45, 60, minutes, 2, 3, 4, 5, 6, 7, 8, 9, 10, 12, 14, 16, 18, 20, 24 hours, 2, 3, 4, 5, 6, 7 days. In another aspect, the first visual cue is selected from a face contour with a region covering the eyes; a cartoon of a face, a cartoon of an animal face, one or more neutral faces with one or more different age ranges or ethnicities; the face of the subject or a family member of the subject; or a trainer. In another aspect, the first visual cue can be one or more images, pre-recorded videos and live video feed from webcam, attached phone, or camera. In another aspect, the processor is programmed with a machine learning algorithm that calculates the landmarks on the face including but not limited to the position of the eyes (eye contour, eyelid, pupil), the focus location on screen, directions of gaze, blinks, dwell time, and/or reciprocal eye engagement with the target's eyes in the training visual cue on computer screen in conjunction with the timestamps of the exposure to the visual cue for one or more eyes of a subject. In another aspect, the one or more visual cue are repeated for 1, 2, 4, 5, 6, 7 days, 1, 2, 3, 4, 5, 6, 7, 8, 9, 10, 11, or 12 weeks, or 1, 2, 3, 4, 5, 6, 7, 8, 9, 10, 11, or 12 months until the dwell time, reciprocal gaze, or both, meets or exceeds the pre-set period of time. In another aspect, the pre-set period of time is 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, or 2.9, 3.0, 3.1, 3.2, 3.3, 3.4, 3.5 seconds. In another aspect, the first, second and additional visual cue(s) is/are, in order: (1) a cartoon of a face or a cartoon of an animal face; (2) one or more neutral faces with one or more different age ranges or ethnicities; (3) the face of the subject or a family member of the subject, and (4) a trainer. In another aspect, the computer or electronic system further comprises using a machine learning algorithm to continuously modify: a length of exposure to one or more visual cues, to increase the dwell time, reciprocal gaze, or both, of the eyes of the subject on the visual cue. In another aspect, the alexithymia is related to the subject having one or more conditions selected from: autism, neurotypical, depression, anxiety, or schizophrenia or a subject with a deficit in recognizing or describing emotions. In another aspect, the training is provided on a computer, laptop, tablet, phone, or other electronic or handheld device.

As embodied and broadly described herein, an aspect of the present disclosure relates to a computer or electronic system for determining a facial emotional analysis, comprising: recording or obtaining one or more images or video of a face of the subject; detecting one or more facial feature in the one or more images or video of the face of the subject at least one of: a contour of the face, a focus location on a screen of one or more eyes, the irises of the one or more eyes, tracking one or more directions of gaze of the one or more eyes, a position of the one or more eyes, one or more blinks, a reciprocal gaze with a first visual cue for one or more eyes, a position of the mouth, a shape of the mouth, one or more eyebrows, a position of the one or more eyebrows, a share of the one or more eyebrows, of the subject in conjunction with exposure to the one or more visual cue cycles; selecting a first visual cue that will engage a visual attention of the subject; using a machine learning algorithm to detect one or more emotions selected from triumph, aesthetic appreciations, relied, pride, admiration, adoration, contentment, satisfaction, love, excitement, interest, awe, amusement, joy, or ecstasy, in the facial features to generate a facial emotion analysis.

### BRIEF DESCRIPTION OF THE DRAWINGS

For a more complete understanding of the features and advantages of the present invention, reference is now made to the detailed description of the invention along with the accompanying figures and in which:

FIG. 1 is a flowchart that outlines the overall system and method of the present invention.

FIG. 2 shows the subjects gender distribution: 8 female and 38 male.

FIG. 3 shows the subjects age distribution between 6 to 13 years old.

FIG. 4 shows the training day with morning and afternoon sessions.

FIG. 5 shows the session, which in this example, involves three cycles, 3s gaze followed by 7s rest per cycle.

FIG. 6 shows, in this example, the 68 facial landmarks and 6 coordinates around the eyes. The facial landmarks can be extended to 400+ data points for comprehensive expression analysis.

FIG. 7 shows the real-time left, right gaze and blink annotation on screen.

FIG. 8 shows the before (left) and after (right) trainer/trainee eye region alignment. The video image represents the trainer, and the green polygons represent the trainee's eye contour. Then, system track reciprocal eye engagements between the trainer and trainee's image frames.

FIG. 9 shows the pre-training images of face contours with blue eye region.

FIG. 10 shows the Level 1 training cartoon images with pupils.

FIG. 11 shows the Level 2 training neutral faces of different age ranges and ethnicities.

FIG. 12 shows the Level 3 self-video and just-in-time feedback annotation on screen.

FIG. 13 shows the Level 4 trainee's eye annotation was overlaid on top of trainer's video to track the reciprocal eye engagement.

FIG. 14 is a flowchart that outlines the overall system and method of the present invention, which includes training levels for eye engagement.

FIG. 15 is a flowchart that outlines a detailed description of one example of the system and method of the present invention, which includes training levels for eye engagement.

FIG. 16 shows a radar diagram presenting the complexity of Hume AI. Most of the emotion AI models recognize positive emotion as happy.

FIG. 17 shows the detection of facial landmarks around the eyes.

FIG. 18 shows the facial emotion skills training, in which mouth, eyebrows, and eyes are identified as the critical features for emotion detection and their facemesh are extracted to recognize the corresponding emotion.

FIG. 19 is a graph that summarizes surprised face face_blendshapes categories and values.

FIG. 20 is a graph that summarizes neutral face face_blendshapes categories and values.

FIG. 21 is a graph that summarizes stressed.

FIG. 22 is a graph that summarizes smile.

FIG. 23 is a graph with data from a specific subject, surprised-Subject #53.

FIG. 24 is a graph with data from a specific subject, neutral-Subject #53.

FIG. 25 is a graph with data from a specific subject, smile-Subject #53.

FIG. 26 is a graph with data from a specific subject, stressed-Subject #53.

FIG. 27 shows an example of forming face tessellation.

FIG. 28 shows an example of detection of the face contours.

FIG. 29 shows an example of detection of the irises.

FIG. 30 compares results for eye tracking using the present invention comparing the dwell time.

FIG. 31A shows the decomposition of facial emotions for 13 selected facial features.

FIG. 31B is a graph and chart that shows the results for the 13 essential facial features selected that are highly relevant to emotion expression.

FIGS. 32A to 32D are graphs that show the reciprocal eye engagement: 3 out of 4 participant subtypes demonstrated statistically significant improvement with p-values 0.027, 0.001, 0.158, and 0.001 respectively.

FIGS. 33A to 33C are graphs that show the facial emotion response over time (weeks 1, week 4, and week 8). All 4 subtypes showed statistically significant improvement with digital twin.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The present invention provides systems and methods for treating or improving neurodivergent conditions by using machine learning to drive human-machine interactions, where eye-gaze-based input systems can offer more natural interaction approaches, particularly for impaired populations.

The present invention provides an effective eye engagement training to enhance dyadic behavior. For example, neurodivergent individuals were able to improve eye engagement after a series of training sessions of progressive difficulty levels. The individuals who have gone through eye engagement training are then able to receive facial emotional cues and be ready for emotional training to alleviate their respective symptoms in Autism Spectrum Disorder (ASD), depression, anxiety, and schizophrenia.

Achieving emotional understanding begins with the ability to focus on individual's eyes. The present inventor recognized that reciprocal eye engagement serves as a gateway to foster dyadic behavior essential for nurturing social relationship. Using machine learning a novel methodology was developed that provides an effective eye engagement training that enhances dyadic behavior and promotes mutual understanding across different individuals in a cohort.

Advantages of the present invention include that it is Personalized Training: Self paced multi-level training based on personal preferences (geometry preference, animal preference, hyperlexia, others). Digital twin training partners to represent self, family members and therapists to reduce stimuli, bridge the gap between self intention and others' perception, and practice the mirror neuron system.

The present disclosure overcomes the problems with existing methods that fail to take into account the differences in the data streams, the ability of the subject to even engage with the images, and fails to provide a personalized method of knowing the reaction, gaze, dwell time, etc., of the specific subject or be able to adapt to the progression of the user during the course of treatment. Existing AI/ML systems are unable to do with by processing real-time images of the subject, and/or provide the specificity needed for individual users. The present disclosure provides novel AI/ML methods for processing video images of an individual, personalizing the data from the same, and then applying a treatment, and tracking progress of the same.

As used herein, the term “digital twin” refers to a 3D model virtual representation of a real person that resembles the real person's facial characteristics and emotion expressions. Trainees can watch the digital twins' videos for the eye engagement training and mimic digital twins' facial expressions for the emotion training. Also, the digital twins can mirror trainee's facial expressions.

The Precision Care provided by the present invention also includes: (1) building a unique multimodal dataset to represent neurodivergent individuals' facial features, including but not limited to 3D tessellations and facial features; (2) computer vision-based AI co-pilot feedback to augment neurological dysfunction; and (3) describing facial emotion expressions in measurable terms using selected facial features, such as mouthSmileLeft, mouthSmileRight, mouthUpperUpLeft, mouthUpperUpRight, brow InnerUp, eyeSquintLeft, eyeSquintRight.

Study. First, the Children's Alexithymia Measure (CAM) were used to measure alexithymia. A total of 10 subjects with clinical assessment of mild ASD symptoms and alexithymia were recruited with ages ranging from 7 to 10. The training was conducted in 4 difficulty levels: cartoon's face, person's still face, and to trainer-led reciprocal eye engagement video sessions. The average optimal eye contact is 3.3 seconds (3.3+/−0.5) [8]. Therefore, this study instructed the training of 3 seconds gaze time. Within each gaze period, this study measured subjects' centered gaze dwell time, looking left, right, and #of blinks. During level 4 training, the occurrence of reciprocal eye engagement was also measured.

It is known that the dorsal parietal cortex is less active when a person with autism tried to maintain eye contact with their partner. The more severe the ASD diagnosis, the less their brain lit up during stimulus [9]. Therefore, it takes tremendous effort and time to improve brain activity toward neurotypical (NT) behavior. Given the difficulty in interacting with an individual therapist, the present invention used a self-paced training to light up the dorsal parietal cortex area of the brain of the subject with ASD.

It was found that after just 4-8 weeks of self-paced training, 6 subjects made statistically significant improvement on both gaze dwell time and number of reciprocal eye engagement. Aggregating all 10 subjects, the improvement on reciprocal eye engagement achieved p-value<5%.

Significance of this study are highlighted below.

(1) Just-In-Time feedback in word form written on screen strengthened the association between eye contact and positive social interactions. This association is typically missing for individuals with alexithymia.

(2) Higher spatial and temporal resolution were obtained via the innovative built-in eye-tracking system compared with the self-reported or experimenter's observation-based measurement, with machine learning used with head-mounted or desk-mounted eye-tracking system to measure eye contact.

(3) Individual-paced progressive training leads to increased engagement, confidence, and better skill retention.

(4) Emphasis on trainer-led reciprocal eye engagement with the two video streams, trainer and subject, overlaying on top of each other achieved an effective reciprocal analysis.

(5) Scalable training sessions were more effective and with a reduced cost. Transcending the physical boundaries via life video cameras, the machine learning assessor of eye gaze was used to eliminate trainer-led therapy to also increase effectiveness and save cost. Once the subject was able to increase reciprocal eye engagement through the first three phases, then the trainer-led therapy was used to further enhance the training.

### Related Eye Contact Measurement Techniques.

Eye contact has been in the research in many disciplines: social cognition, social psychology, psychiatry such as autism spectrum disorder, etc. There have been numerous eye contact measurement techniques.

There are two general categories: direct and indirect. Direct measurement refers to those that eye contact is assessed while it occurs and is not retrospectively verifiable. Whereas indirect measures eye contact after it has occurred and is therefore verifiable retrospectively.

Popular techniques used to study eye contact [10]

| Category | Technique | Description |
|---|---|---|
| Direct | Estimation | The occurrence of eye contact-whether it occurred |
| | binary or | (yes/no) or a time estimate (in seconds)-is estimated |
| | in time | |
| | Coding | The occurrence and duration of eye contact is |
| | sheet | registered on a predefined coding sheet |
| | Timer | The occurrence and duration of eye contact is timed |
| | | using a digital timer, such as a stopwatch |
| | Event | The occurrence and duration of eye contact is |
| | recorder | registered using an event recorder, which is activated |
| | | by pressing or depressing a button |
| Indirect | Video | The interaction is registered on either one or multiple |
| | camera | video cameras. The video lens is zoomed either on the |
| | | face, the upper body, or full body/the whole scene. |
| | | The occurrence and duration of eye contact is derived |
| | | from the video registrations |
| | Camera on | The interaction is registered on a small video camera |
| | glasses | located on the nose bridge of a pair of glasses. The |
| | | video camera captures video recordings following the |
| | | orientation of the head. The occurrence and duration |
| | | of eye contact is derived from the video registrations |
| | Eye | The occurrence and duration of eye contact is |
| | tracking: | registered through a head mounted wearable eye |
| | head | tracking device. Gaze location in head-centered |
| | mounted | coordinates is recorded. Gaze location is mapped to |
| | | the image from the scene camera (a camera directed |
| | | towards the world from the participant's head). Eye |
| | | contact is inferred from the gaze location being in a |
| | | specified area (e.g. the eye or face region of another |
| | | person) |
| | Eye | The occurrence and duration of eye contact in a |
| | tracking: | human interaction is registered through a desk |
| | desk | mounted eye tracking device, which is located under |
| | mounted | a computer screen at a distance from the participant. |

The present invention applied cutting-edge computer vision and digital twin technologies and machine learning for eye engagement training and achieved statistically significant improvement based on real-time measurement.

Subjects. A total of 10 subjects, ages ranging from 7 to 10, were recruited who have clinical assessment of mild ASD symptoms and were also assessed with alexithymia using Children's alexithymia Measure (CAM) [11].

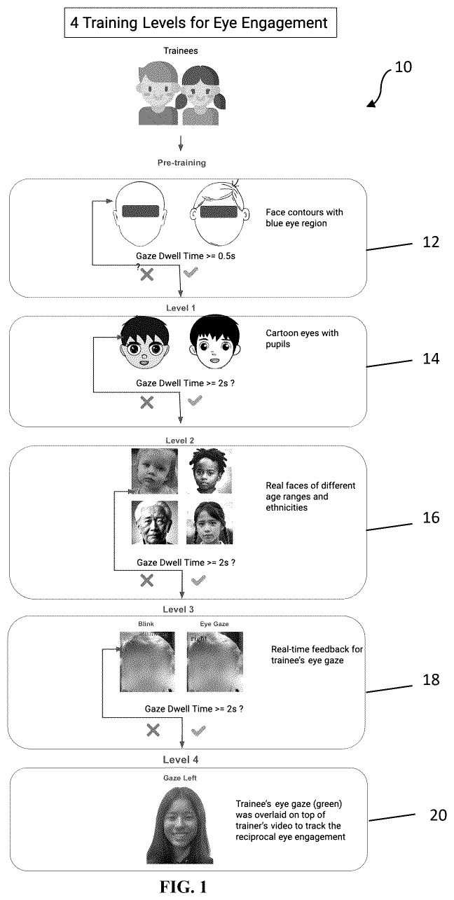

FIG. 1 is a flowchart 10 that outlines the overall system and method of the present invention. The trainees undergo an initial pre-training in step 12 that includes face contours with a blue eye region and dwell time is assessed until the dwell time increase to an average being equal to or greater than 0.5 second. Once the subject achieves this dwell time, in Level 1 (step 14) the subject is transitioned to looking at cartoon eyes with pupils and again the dwell time is measured. Once the subject's dwell time reaches an average being equal to or greater than 2 seconds, then the subject transitions to Level 2 (step 16). In step 16, the subject is transitioned to real faces of different age ranges and ethnicities, and again the subject's dwell time is measured until it reaches an average being equal to or greater than 2 seconds. Next, Level 3 training (step 18) involves looking at real-time feedback for a trainee's eye gaze using images of the subject. In Level 3, the subject can be evaluated for blink and/or eye gaze, and once the subject's blink and/or eye gaze dwell time reaches an average being equal to or greater than 2 seconds, then the subject transitions to Level 4 (step 20). In Level 4, the trainee's eye gaze is overlaid over the top of a video of the trainer to track reciprocal engagement of the subject.

The steps for tracking the reciprocal eye engagement: (1) Computer vision such as mediapipe is used to capture the face landmarks (image points) of nose, chin, left eye left corner, right eye right corner, left mouth corner, right mouth corner. (2) The key points in 3D model of the face (object points) are tagged. (3) Method such as, not limited to, cv2.solvePnP ( ) is used to estimate the orientation of the 3D face in the 2D image. (4) Method such as, not limited to, cv2.estimateAffine3D is used to compute an optimal affine transformation from image points to 3D points for both eyes. (5) Based on each eye pupil's 3D coordinate, compute the project points on computer screen using method such as cv2.projectPoints. (6) Draw project area in trainer's video. (7) Track occurrence of reciprocal eye engagement (#of image frames that reciprocal occurred/total #of image frames trainer gazed center).

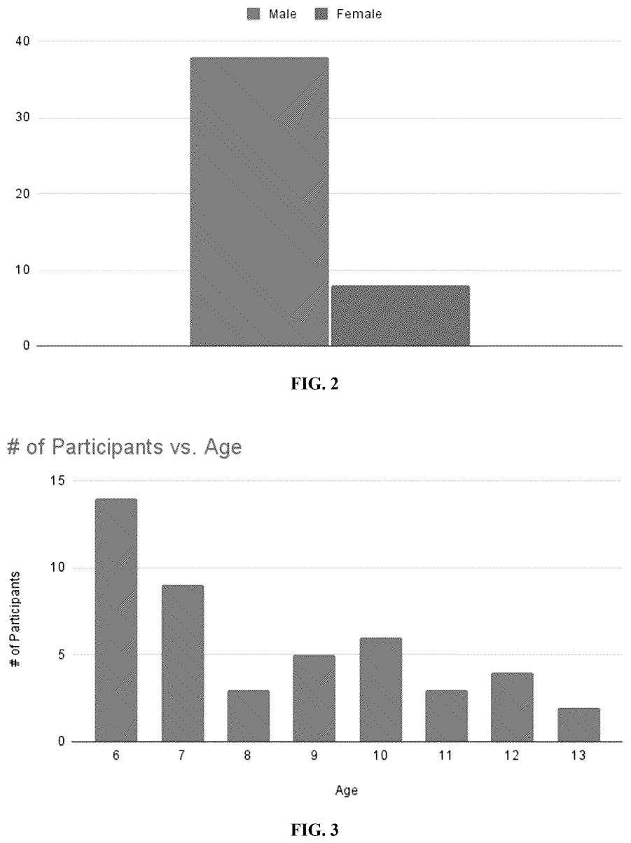

FIG. 2 is a bar-chart that shows the subjects' gender distribution: 8 female and 38 male.

FIG. 3 is a graph that shows the subjects' age distribution between 6 to 13 years old.

### Eye Engagement Training Design.

The scientific study defines the average optimal eye contact as 3.3 seconds (3.3+/−0.5) [8]. Therefore, this study instructs the training of 3 seconds gaze dwell time. For each training day, a subject is trained in two sessions: morning and afternoon.

FIG. 4 shows a graphic with an example of the training day with morning and afternoon sessions. Each session involves three consecutive cycles of training. Due to the discomfort associated with the prolonged gaze, each cycle is subdivided into 3-second watching the eyes on the screen followed by a 7-second resting period.

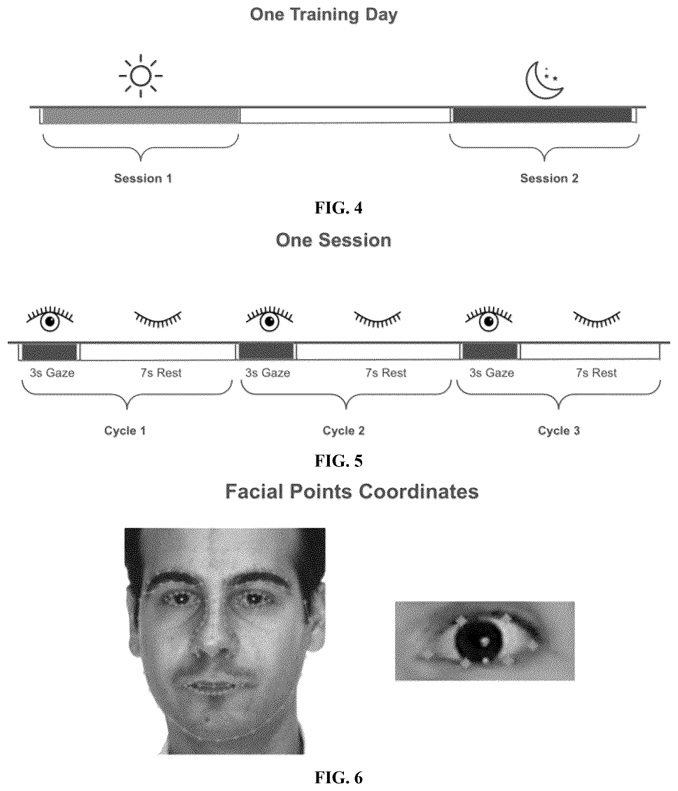

FIG. 5 shows an example of the session involves three cycles, 3s gaze followed by 7s rest per cycle.

### AI-Based Eye Engagement Measurement.

This study applied computer vision and video processing techniques to track the following metrics for each 3-second gaze training period: center gaze dwell time (duration of fixation in center), #of blinks, #of left movement, #of right movement, and for Level 4 only, occurrence of reciprocal eye engagement (both subject and trainer gaze at each other simultaneously).

This study used a series of ML technologies for computer vision and image processing tasks in the following six steps.

### Step 1: Preprocess the Input Video.

OpenCV, a real-time computer vision library, was used to take video of the trainees from webcam (level 1, 2, 3, and 4 training) and the video of the live trainers (level 4).

cv2.VideoCapture(n) (1)

The input parameter number represents the camera index number. It can be a webcam, connected phone camera, and a virtual camera. Once the images were read into a 2D array from the camera, the images in the 2D array were flipped horizontally so that the recognition of left and right eye movements align with the subjects facing the camera. Then the color images were converted into grayscale before being sent to the face detector in step 2.

### Step 2: Locate Face in the Image.

The output of this step is the face bounding box (e.g. the (x, y) coordinates). Technologies, without limitation, Dlib and mediapipe were used for face detection, face recognition and facial landmark detection [12]. It relies on Histogram of Oriented Gradients (HOG)+Linear SVM face detector. It was trained with 7220 images from various datasets like ImageNet, PASCAL VOC, VGG, WIDER, Face Scrub.

This technology handles the face image well when multiple objects come too close or combine with each other, i.e. occlusion. Also, the technology works well for different face orientations. In the conversation lacking effective eye engagement, diverse face orientations are observed. During level 4 training, the trainers can choose to express their mild facial expressions and slight face turns depending on the training progress of the individuals. The technology used does not handle face sizes less than 80×80 due to the training dataset.

Face detector technologies were used to detect a list of faces. An example of such technologies is Dlib function get_frontal_face_detector ( ) This function was called to retrieve a Histogram of Oriented Gradients (HOG)+Linear SVM face detector. Then the preprocessed grayscale images from the video were passed to the face detector to get a list of detected faces.

### Step 3: Detect and Annotate the Eye Regions.

The shape predictor with 68 facial landmarks was applied to each of the detected faces from step 2 above to detect the key facial structures on the face Region of Interest (ROI).

FIG. 6 shows an example of the 68 facial landmarks and 6 coordinates around the eyes.

The following function was called to receive a shape object containing the 68 (x, y)-coordinates of the facial landmark regions. 6 coordinates were allocated for each eye.

shape_predictor(‘shape_predictor_68_face_landmarks.dat’) (2)

Then shape_to_np function was called to convert the shape object to a NumPy array for Python code. During the real-time training, polylines were drawn around the eye regions using the 6 coordinates per eye.

### Step 4: Detect and Display Center Gaze Dwell, Left and Right Eye Movement.

First, system constructed the eye image using eye region coordinates along with the grayscale face image. Then the eye image was sent to the “gaze” model to predict the gaze events: center, left and right. The result was real-time annotated on screen as a feedback signal for trainees. At the same time, the result was written into the system log for retrospective learning.

### Step 5: Detect and Display Blink.

The eye image was sent to the “blinkdetection” model to predict the blink event. The result was real-time annotated on screen as a feedback signal as well as written into log for retrospective learning.

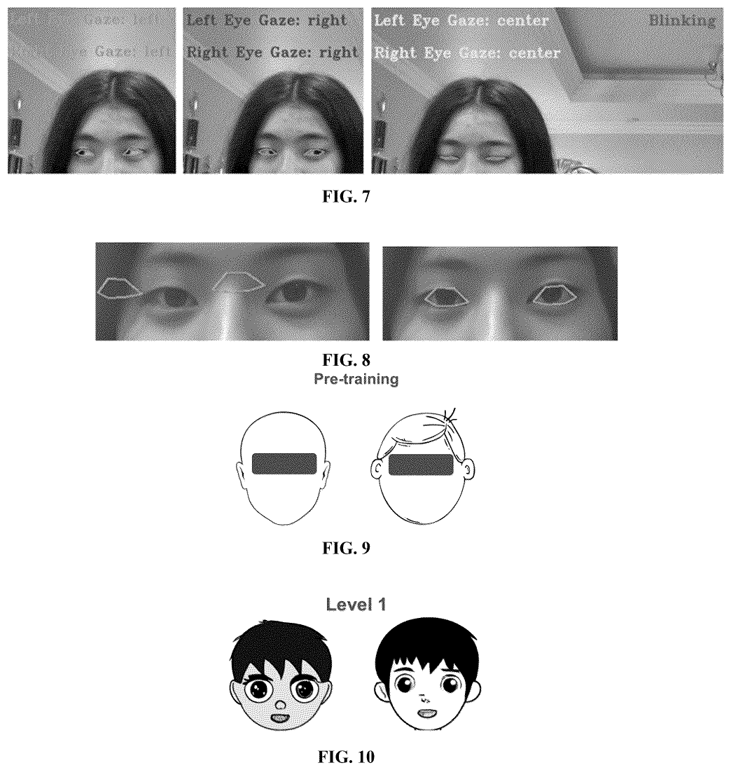

FIG. 7 shows an example of the Real-time left, right gaze and blink annotation on screen.

With the eye regions and gaze annotation, the feedback from some subjects was that they gained a sensation of control and power with their eye gaze.

### Step 6: Reciprocal Eye Engagement.

For level 4 trainer-led session, system tracks the occurrence of reciprocal eye engagement when both subject and trainer gaze at each other simultaneously. Two cameras were used:

Camera for trainer. The full video was displayed at the center of the screen for subject to watch and interact with.

Camera for trainee. This is the webcam on laptop because subject needs to look at the trainer video on the laptop screen and the gaze direction needs to be aligned for system analysis. Only the subject's eye regions (green polygons) were displayed on screen overlaying on top of the trainer's video.

Inside the system, the two video streams overlaid on top of each other. Trainer's full video was displayed, and only subject's eye region boxes were displayed. First, calibration was done to align subject's eye regions with trainer's eye regions on screen. The steps for tracking the reciprocal eye engagement: (1) Computer vision such as mediapipe is used to capture the face landmarks (image points) of nose, chin, left eye left corner, right eye right corner, left mouth corner, right mouth corner. (2) The key points in 3D model of the face (object points) are tagged. (3) Method such as, not limited to, cv2.solvePnP ( ) is used to estimate the orientation of the 3D face in the 2D image. (4) Method such as, not limited to, cv2.estimateAffine3D is used to compute an optimal affine transformation from image points to 3D points for both eyes. (5) Based on each eye pupil's 3D coordinate, compute the project points on computer screen using method such as cv2.projectPoints. (6) Draw project area in trainer's video. (7) Track occurrence of reciprocal eye engagement (#of image frames that reciprocal occurred/total #of image frames trainer gazed center).

FIG. 8 shows an example of the before (left) and after (right) trainer/trainee eye region alignment. The video image represents the trainer, and the green polygons represent the trainee's eye regions. Then, system tracked reciprocal eye engagements between the two image frames.

Training Process and administering a treatment based on the recorded tracking by the tracking system above.

This study introduced an individual-paced progressive training approach with one pre-training assessment and four levels of training. This approach is important because eye contact maintenance is not merely a challenge in the behavior, but deeply rooted in neurodevelopment. Each subject took the number of days they needed, up to 2 weeks per training level. This leads to increased engagement, confidence, and better skill retention.

Participants were recorded with an AI-based eye-tracking system and laptop webcam during each of the training levels. The video lens was zoomed on the face or the upper body.

Pre-Training Assessment. Studies on the gaze aversion model find that when looking at eyes, emotion-processing regions of the brain, such as the amygdala, are more active for autistic individuals compared to neurotypicals [15]. This is reflected in their behavior: to reduce sensory overload, many autistics will not make eye contact when listening to their partners' talking.

As the prerequisite of this training, subjects need to be able to recognize and gaze at the eye area of the face for at least 0.5 seconds. To reduce the stimuli of the eyes, face contours were displayed on the screen with highlighted blue eye region. It was found that just 2 of the 10 subjects took additional days to qualify this pre-training assessment.

FIG. 9 shows an example of the pre-training images of face contours with blue eye region.

### Level 1: Cartoon Faces.

Cartoon eyes with pupils on a neutral face were introduced as training images.

FIG. 10 shows an example of the Level 1 training cartoon images with pupils.

### Level 2: Real Human Faces.

Research has found that elevated amygdala responses are associated with eye gaze on a neutral face. This suggests increased arousal for face stimuli [14].

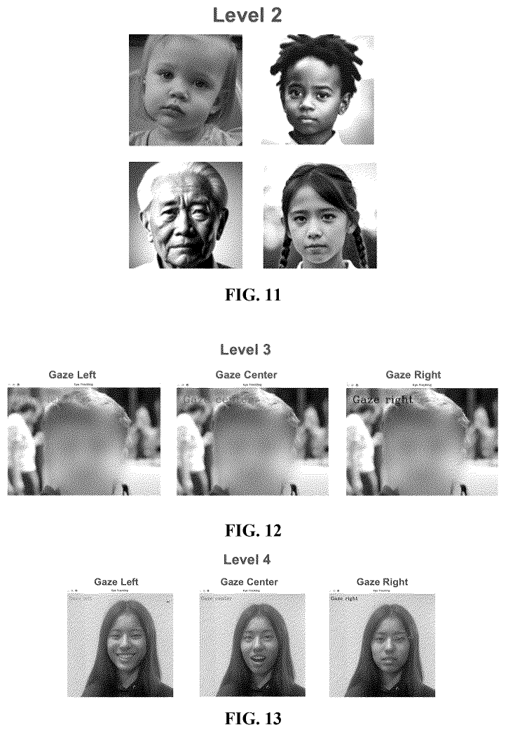

FIG. 11 shows an example of the Level 2 training neutral faces of different age ranges and ethnicities.

### Level 3: Real-Time Feedback on Self-Video.

With the distracting factor of constantly changing video frames, it adds another layer of challenge. Some studies have found that in autism, since the amygdala does not respond the same way, autistic individuals' brains don't learn to make the same strong associations between eye contact and positive social interactions. Therefore, this study designed the UI to provide just-in-time feedback for subject's eye gaze during the training.

FIG. 12 shows an example of the Level 3 self-video and just-in-time feedback annotation on screen.

TABLE 1
sample eye gaze measurement for subjectid 003.
SubjectId: 003

| | Training | Gaze Dwell | # Blinks in | Total # | # Moving | # Moving |
|---|---|---|---|---|---|---|
| Day/Session | Cycle # | Time (s) | Gaze Period | of moves | Left | Right |
| Day 1/Session 1 | 1 | 0.5 | 2 | 2 | 2 | 0 |
| Session 2 | 2 | 1 | 1 | 2 | 1 | 1 |
| | 3 | 0.9 | 2 | 0 | 0 | 0 |
| | 4 | 2 | 0 | 1 | 0 | 1 |
| | 5 | 1.7 | 7 | 1 | 0 | 1 |
| | 6 | 1.8 | 1 | 1 | 1 | 0 |
| Day 2/Session 1 | 7 | 1.2 | 0 | 2 | 1 | 1 |
| Session 2 | 8 | 1.7 | 1 | 2 | 1 | 1 |
| | 9 | 1.9 | 2 | 0 | 0 | 0 |
| | 10 | 2.1 | 0 | 1 | 0 | 1 |
| | 11 | 2.4 | 2 | 1 | 0 | 1 |
| | 12 | 1.8 | 1 | 1 | 1 | 0 |
| Day 3/Session 1 | 13 | 1.6 | 3 | 1 | 0 | 1 |
| Session 2 | 14 | 2 | 1 | 1 | 1 | 0 |
| | 15 | 1.9 | 1 | 0 | 0 | 0 |
| | 16 | 2.1 | 2 | 0 | 0 | 0 |
| | 17 | 2.6 | 0 | 1 | 0 | 1 |
| | 18 | 2 | 1 | 1 | 1 | 0 |

### Level 4: Trainer-Led Reciprocal Eye Engagement Training.

This level of training resembles real life interactions (i.e. reciprocal eye contact). Research in non-autistics shows that when two people converse, their eye contact periodically synchronizes, signifying shared attention [15] [16]. In contrast, autistics don't usually sync eye contact. For example, to reduce sensory overload, many autistics will make eye contact when talking, but not when listening. One explanation for this pattern of behavior is that due to sensory overload, it is more difficult to concentrate on auditory information while also looking at someone's eyes [15].

The subjects were being watched by the trainer with the trainer attempting to exchange facial expressions and leading the reciprocal eye engagement practice via another real-time video-camera. The goal of this level was to enhance the joined attention and dyadic behavior. Please refer to step-6 in section “AI-Based Eye Engagement Measurement” for details.

FIG. 13 shows an example of the Level 4 trainee's eye annotation was overlaid on top of trainer's video to track the reciprocal eye engagement.

### Observations and Result Analysis

Subjects gazed less to the eye areas in level 4 training when the trainer watched them with the attempt to exchange facial emotional expressions. At the beginning of each level, subjects experienced a slight degradation in eye gazing comparing with the end of prior level. The improvement rate did not correlate with subjects' gender and age.

It was found that 6 out of 10 subjects made statistically significant improvement over the self-paced training period between 4-8 weeks with p-value<5% for both gaze dwell time and number of reciprocal eye engagement.

In addition, 3 subjects showed improvements on gaze dwell time over the period of 8 weeks. 1 subject showed no improvement.

Following table shows the statistical analysis results. Each row represents one eye-tracking measurement. Column 1-6 represents each subject respectively and the numeric values in these columns show the correlation scores between the corresponding measurement with the time progression. E.g. the higher correlation score for “Gaze Dwell Time” means the gaze dwell improved over time for the given subject. The negative correlation score means that the given measurement number reduced overtime. The right two columns represent the statistical significance value (p-value) for the given eye-tracking measurement when aggregating all the subjects.

The result shows statistically significant improvement on gaze dwell time (p-value=0.000931) and reciprocal eye engagement (p-value=0.021000). The #of blinks did not show significant improvement.

TABLE 2
Statistical analysis for 6 subjects and their aggregated p-value.

| | 1 | 2 | 3 | 4 | 5 | 6 | sig_inc | sig_dec |
|---|---|---|---|---|---|---|---|---|
| Cycle # | 1.000000 | 1.000000 | 1.000000 | 1.000000 | 1.000000 | 1.000000 | 0.000000 | 1.000000 |
| Gaze Dwell Time (s) | 0.379400 | 0.139700 | [symbol] | [symbol] | [symbol] | [symbol] | 0.000931 | 0.999069 |
| # Blinks in Gaze Period | 0.154500 | 0.234000 | −0.142300 | [symbol] | 0.060500 | 0.234000 | 0.000931 | 0.999059 |
| Total # of moves | −0.270700 | 0.083200 | −0.270700 | −0.256100 | 0.065700 | −0.104500 | 0.937333 | [symbol] |
| # Moving Left | −0.071600 | 0.159500 | −0.071600 | −0.326800 | 0.127200 | 0.166400 | 0.513564 | 0.486436 |
| # Moving Right | −0.286400 | −0.055500 | −0.286400 | 0.309600 | −0.078400 | −2.86400 | 0.856901 | 0.143099 |
| Reciprocal Eye Engagement | 0.129400 | 0.443000 | 0.345500 | [symbol] | [symbol] | −0.151900 | 0.021000 | 0.979000 |

[symbol]

indicates data missing or illegible when filed

Novel personalized training system to improve reciprocal eye engagement and facial emotional skills for neurodivergent individuals using computer vision, multimodal models and digital twins.

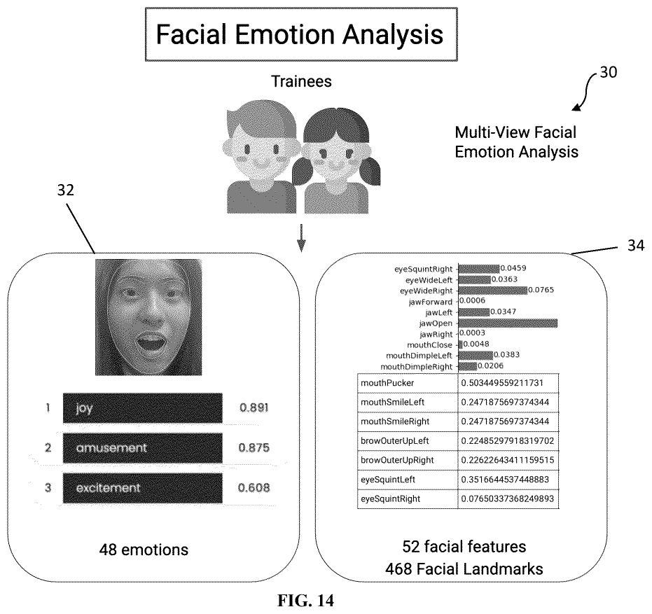

As shown in FIG. 14, is a flowchart 30 that outlines the overall system and method of the present invention, which includes training levels for eye engagement. The system can also be used to achieve multi-label recognition of, e.g., in step 32-48 emotions can be measured as well as, in step 34-52 facial features 34 and 468 facial landmarks.

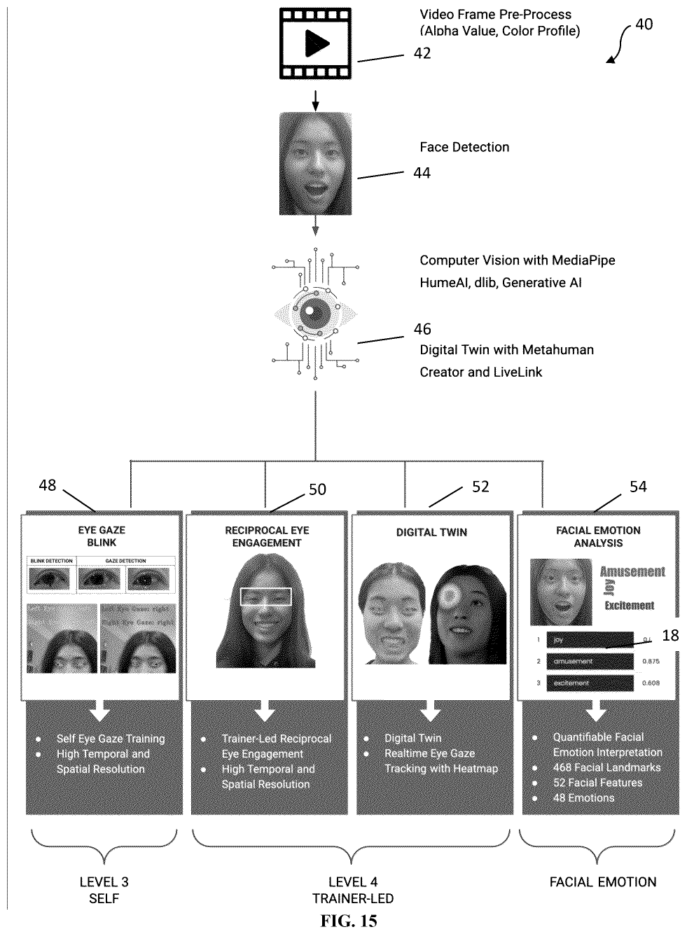

FIG. 15 is a flowchart 40 that outlines a detailed description of one example of the system and method of the present invention, which includes training levels for eye engagement. First, in step 42, the video frame pre-processing (e.g., alpha value, color profile) are obtained. Next, in step 44 the face is detected. In step 46, a digital twin of the subject is created as outlined hereinbelow. Finally, the method is used for Level 3 training (step 48) as described hereinabove, in Level 4 trainer-led training (step 50 and 52), and finally it can be used for facial emotion analysis (step 54). Each of the steps 42, 44, and 46 are described in detail below.

Eye gaze and blink analysis. Video frame pre-processing (step 42) can include eye gaze and blink analysis. MediaPipe is one of the technologies that can be used for eye gaze, dwell time and blink tracking. Mediapipe an open source ML framework that orchestrates multimodal models. This research was built on top of the 2023 new release on Tasks, specifically the features on computer visions. (https://developers.google.com/mediapipe). Most MediaPipe features use different TFLite models (TensorFlow Lite). MediaPipe version in colab environment: Mediapipe-0.10.9-cp310-cp310-manylinux_2_17_x86_64

Pre-process Image on Alpha Value and sRGB Color Profile. Remove alpha value. In digital images, each pixel contains RGB values describing the intensity of red, green and blue. Each pixel also contains an Alpha value describing its opacity. An alpha value of 1 means totally opaque and 0 means totally transparent. Before the image is sent to the ML pipeline, the alpha value is removed. Convert color profile to SRGB, this example used color profile SRGB (mediaPipe.ImageFormat.SRGB), but equivalent systems may be used.

sRGB is a standard RGB (red, green, blue) color space that HP and Microsoft created cooperatively in 1996 to use on monitors, printers, and the World Wide Web. It was subsequently standardized by the International Electrotechnical Commission (IEC) as IEC 61966-2-1:1999. (webstore.iec.ch/publication/6169).

Using sRGB as the default color profile is a good choice when designing for web, or for a wide variety of displays. Modern web browsers follow W3C standards and use sRGB as their color profile. All phones, Macs and most screens can display these colors.

Display P3 is better if work contains many videos or photos, and make use of more vibrant colors when designing for devices that support the Display P3 color profile (like all modern iPhones). Photos captured by iPhone have this color profile.

| | | | |
|---|---|---|---|
| ∨ More Info: | | ∨ More Info: | |
| Last opened: | Feb. 9, 2024 at 10:30 AM | Where from: https:// | |
| Dimensions: | 2316 × 3088 | | colab.research.google.com/ |
| Device make: | Apple | | blob:null/8325df7f- |
| Device model: | iPhone XR | | bf3e-453c-98b8- |
| Color space: | RGB | | ee206e4ab7b4 |
| Color profile: | Display P3 | Last opened: | Feb. 9, 2024 at 10:32 AM |
| Focal length: | 2.87 mm | Dimensions: | 958 × 1358 |
| Alpha channel: | Yes | Color space: | RGB |
| Red eye: | No | Color profile: | sRGB IEC61966-2.1 |
| Metering mode: | Pattern | Alpha channel: | No |
| F number: | f/2.2 | | |
| Exposure program: | Normal | ∨ Name & Extension: | |
| Exposure time: | 1/30 | | |
| Latitude: | 37° 18′ 18.828″ N | image.png | |
| Longitude: | 122° 2′ 19.31″ W | □ Hide extension | |

For example, an image is represented with a list of RGB values.
[[−31.20965738] #pixel 1 R value
[33.07340551] #pixel 1 G value
[−26.01151735]] #pixel 1 B value
[−29.28600113] #pixel 2 R value
[32.78101752] #pixel 2 G value
[−26.00249889]] #pixel 2 B value

Face Detection is described in connection with step 44. System identifies the face area in the image and sends the face image to the following computer vision pipeline. An example of code is as follows:

```
Create MediaPipe Image import cv2
cap = cv2.VideoCapture(0)
while cap.isOpened( ):
  success, image = cap.read( ) If not success:
    break
  rgb_frame = mp.Image(image_format=mp.ImageFormat.SRGB, data=image)
```

Facial Landmark Detector. An example of code for creating a Facial Landmark Detector based on MediaPipe Computer Vision model.

Create model asset ″face_landmarker_v2_with_blendshapes.task″
base_options =
python.BaseOptions(model_asset_path=″face_landmarker_v2_with_blendshapes.task″)
options = vision.FaceLandmarkerOptions(base_options=base_options,
output_facial_transformation_matrixes=True,
detector = vision.FaceLandmarker.create_from_options(options)

Detect and Scale Facial Landmark Positions to Image Coordinates. For the real-time detected 468 3D facial landmarks, the system retrieves the normalized x/y/z values in range [0.0, 1.0].

The system uses the normalized landmarks to represent a point in 3D space with x, y, z coordinates. x and y are normalized to [0.0, 1.0] by the image width and height respectively. z represents the landmark depth, and the smaller the value the closer the landmark is to the camera. The magnitude of z uses roughly the same scale as x.

### Option 1: Manually Scale in the Code

Following code scales the normalized x value between [0.0, 1.0] to the x coordinate on image between [0.0, image.shape [1]]. Here, image.shape [1] represents the image width.

$$x = int ( landmark . x * image . shape [ 1 ] )$$

Similarly, the following code scales the normalized y value to y coordinate on image. Image.shape [0] represents the image height.

```
y = int(landmark.y * image.shape[0])
Option 2: Automatic scale using face_landmarks_proto
  face_landmarks_proto = landmark_pb2.NormalizedLandmarkList( )
  face_landmarks_proto.landmark.extend([
    landmark_pb2.NormalizedLandmark(x=landmark.x, y=landmark.y, z=landmark.z) for
landmark in face_landmarks
  ]
)
```

### Step 5. Annotate Landmarks on Face Image.

Option 1: Manually draw the landmarks on image. This gives more flexibility in drawing style. #draw green dots with dimension 2.
cv.circle (image, (x, y), 2, (0, 255, 0), −1)

### Option 2: Automatically Draw on Image Using MediaPipe Drawing Utilities.

solutions.drawing_utils.draw_landmarks(
image=annotated_image,
landmark_list=face_landmarks_proto,
connections=mp.solutions.face_mesh.FACEMESH_IRISES,
landmark_drawing_spec=None,
connection_drawing_spec=DrawingS₁

Multimodal Facial Emotion Analysis. Computer vision and facial emotion recognition AI models such as MediaPipe (explained above) and Hume AI are used for this module. Hume AI model specializes in understanding and classifying emotional expression from text, images, videos, and audio clips. This model expands to 48 emotion categories and effectively supports multi-label classification design. It outputs multiple emotions for each image, truly representing the complexity of actual human emotions.

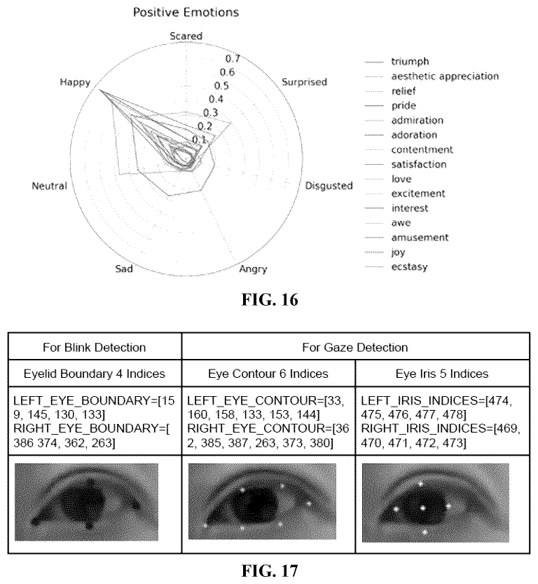

FIG. 16 shows a radar diagram presenting the complexity of Hume AI. Most of the emotion AI models recognize positive emotion as happy. Hume AI further categorizes them as triumph, aesthetic appreciation, relief, pride, admiration, adoration, contentment, satisfaction, love, excitement, interest, awe, amusement, joy, and ecstasy.

Step 46 includes the creation of a personalized digital twin as the trainer partner. A digital twin is a 3D model virtual representation of a real person that resembles the real person's facial characteristics and emotion expressions. Research shows that training eye engagement and facial emotions with digital twins pose less stress on the neurodivergent individuals and is an effective approach towards adapting to real-world scenarios. Trainees can watch the digital twins' videos for the eye engagement training and mimic digital twins' facial expressions for the emotion training. Also, the digital twins can mirror trainee's facial expressions.

### Eye Engagement Training Level 1, 2, 3 (Gaze, Dwell, Blink).

FIG. 17 shows the detection of facial landmarks around the eyes. The method uses eyelid boundary landmarks to identify blink and uses eye contour and eye iris landmarks to identify gaze direction and duration.

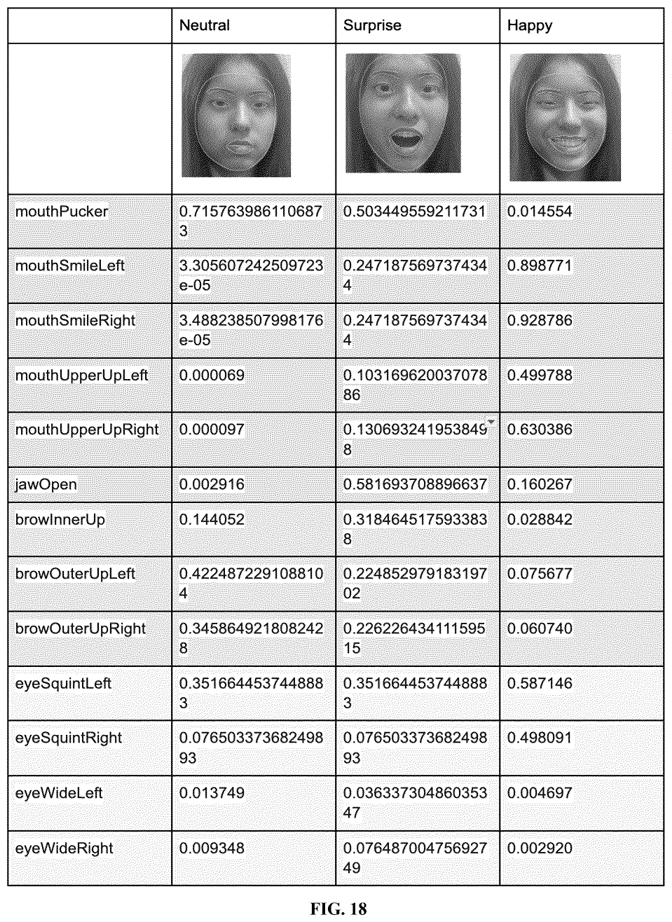

Eye Engagement Training Level 4 (Trainer-Led Reciprocal Eye Engagement). FIG. 18 shows the facial emotion skills training, in which mouth, eyebrows, and eyes are identified as the critical features for emotion detection and their facemesh are extracted to recognize the corresponding emotion.

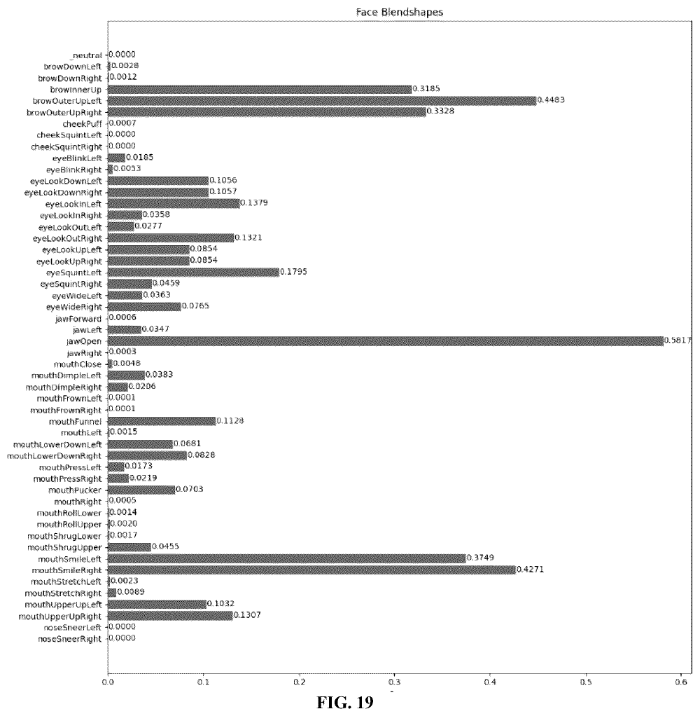

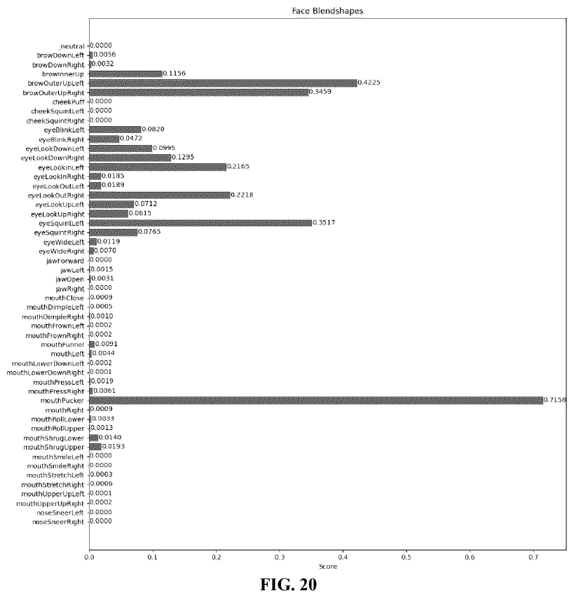

FIG. 19 is a graph that summarizes surprised face face_blendshapes categories and values.

Code for determining faceblend can be, e.g.:
for face_blendshapes_category in face_blendshapes: print (face_blendshapes_category)

### Surprised:

| Category(index=0, | score=3.398218950678711e−06, | display_name=”, |
|---|---|---|
| category_name=″_neutral″) | | |
| Category(index=1, | score=0.0028309973422437906, | display_name=”, |
| category_name=″browDownLeft″) | | |
| Category(index=2, | score=0.0011817640624940395, | display_name=”, |
| category_name=″browDownRight″) | | |
| Category(index=3, | score=0.3184645175933838, | display_name=”, |
| category_name=″browInnerUp″) | | |
| Category(index=4, | score=0.4483375549316406, | display_name=”, |
| category_name=″browOuterUpLeft″) | | |
| Category(index=5, | score=0.3327658176422119, | display_name=”, |
| category_name=″browOuterUpRight″) | | |
| Category(index=6, | score=0.0006742323166690767, | display_name=”, |
| category_name=″cheekPuff″) | | |
| Category(index=7, | score=1.7782591612558463e−06, | display_name=”, |
| category_name=″cheekSquintLeft″) | | |
| Category(index=8, | score=1.1791496490332065e−06, | display_name=”, |
| category_name=″cheekSquintRight″) | | |
| Category(index=9, | score=0.018461165949702263, | display_name=”, |
| category_name=″eyeBlinkLeft″) | | |
| Category(index=10, | score=0.005333852022886276, | display_name=”, |
| category_name=″eyeBlinkRight″) | | |
| Category(index=11, | score=0.10564041137695312, | display_name=”, |
| category_name=″eyeLookDownLeft″) | | |
| Category(index=12, | score=0.10567165166139603, | display_name=”, |
| category_name=″eyeLookDownRight″) | | |
| Category(index=13, | score=0.13789816200733185, | display_name=”, |
| category_name=″eyeLookInLeft″) | | |
| Category(index=14, | score=0.03575790300965309, | display_name=”, |
| category_name=″eyeLookInRight″) | | |
| Category(index=15, | score=0.02766125649213791, | display_name=”, |
| category_name=″eyeLookOutLeft″) | | |
| Category(index=16, | score=0.13205760717391968, | display_name=”, |
| category_name=″eyeLookOutRight″) | | |
| Category(index=17, | score=0.0853993371129036, | display_name=”, |
| category_name=″eyeLookUpLeft″) | | |
| Category(index=18, | score=0.08544529229402542, | display_name=”, |
| category_name=″eyeLookUpRight″) | | |
| Category(index=19, | score=0.17954489588737488, | display_name=”, |
| category_name=″eyeSquintLeft″) | | |
| Category(index=20, | score=0.045866597443819046, | display_name=”, |
| category_name=″eyeSquintRight″) | | |
| Category(index=21, | score=0.03633730486035347, | display_name=”, |
| category_name=″eyeWideLeft″) | | |
| Category(index=22, | score=0.07648700475692749, | display_name=”, |
| category_name=″eyeWideRight″) | | |
| Category(index=23, | score=0.0005817460478283465, | display_name=”, |
| category_name=″jawForward″) | | |
| Category(index=24, | score=0.03474847599864006, | display_name=”, |
| category_name=″jawLeft″) Category(index=25, score=0.581693708896637, | | display_name=”, |
| category_name=″jawOpen″) Category(index=26, score=0.00034396781120449305, | | display_name=”, |
| category_name=″jawRight″) | | |
| Category(index=27, | score=0.0048374151811003685, | display_name=”, |
| category_name=″mouthClose″) | | |
| Category(index=28, | score=0.03832445293664932, | display_name=”, |
| category_name=″mouthDimpleLeft″) | | |
| Category(index=29, | score=0.020560793578624725, | display_name=”, |
| category_name=″mouthDimpleRight″) | | |
| Category(index=30, | score=8.363015513168648e−05, | display_name=”, |
| category_name=″mouthFrownLeft″) | | |
| Category(index=31, | score=0.000109163585875649, | display_name=”, |
| category_name=″mouthFrownRight″) | | |
| Category(index=32, | score=0.11284423619508743, | display_name=”, |
| category_name=″mouthFunnel″) | | |
| Category(index=33, | score=0.0014914023922756314, | display_name=”, |
| category_name=″mouthLeft″) | | |
| Category(index=34, | score=0.06805182993412018, | display_name=”, |
| category_name=″mouthLowerDownLeft″) | | |
| Category(index=35, | score=0.08284611999988556, | display_name=”, |
| category_name=″mouthLowerDownRight″) | | |
| Category(index=36, | score=0.017273977398872375, | display_name=”, |
| category_name=″mouthPressLeft″) | | |
| Category(index=37, | score=0.02186986431479454, | display_name=”, |
| category_name=″mouthPressRight″) | | |
| Category(index=38, | score=0.07025376707315445, | display_name=”, |
| category_name=″mouthPucker″) | | |
| Category(index=39, | score=0.0005339805502444506, | display_name=”, |
| category_name=″mouthRight″) | | |
| Category(index=40, | score=0.0013860255712643266, | display_name=”, |
| category_name=″mouthRollLower″) | | |
| Category(index=41, | score=0.0019505214877426624, | display_name=”, |
| category_name=″mouthRollUpper″) | | |
| Category(index=42, | score=0.0016738412668928504, | display_name=”, |
| category_name=″mouthShrugLower″) | | |
| Category(index=43, | score=0.04552755877375603, | display_name=”, |
| category_name=″mouthShrugUpper″) | | |
| Category(index=44, | score=0.3749385476112366, | display_name=”, |
| category_name=″mouthSmileLeft″) | | |
| Category(index=45, | score=0.4270780086517334, | display_name=”, |
| category_name=″mouthSmileRight″) | | |
| Category(index=46, | score=0.0023029844742268324, | display_name=”, |
| category_name=″mouthStretchLeft″) | | |
| Category(index=47, | score=0.008859497494995594, | display_name=”, |
| category_name=″mouthStretchRight″) | | |
| Category(index=48, | score=0.10316962003707886, | display_name=”, |
| category_name=″mouthUpperUpLeft″) | | |
| Category(index=49, | score=0.1306932419538498, | display_name=”, |
| category_name=″mouthUpperUpRight″) | | |
| Category(index=50, | score=3.400102286832407e−06, | display_name=”, |
| category_name=″noseSneerLeft″) | | |
| Category(index=51, | score=4.805532171303639e−06, | display_name=”, |
| category_name=″noseSneerRight″) | | |

FIG. 20 is a graph that summarizes neutral face face_blendshapes categories and values.

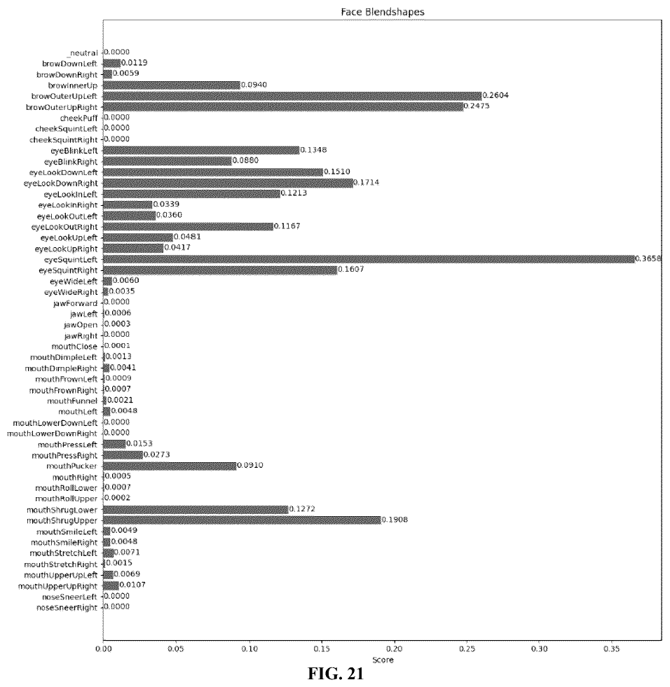

FIG. 21 is a graph that summarizes stressed.

FIG. 22 is a graph that summarizes smile.

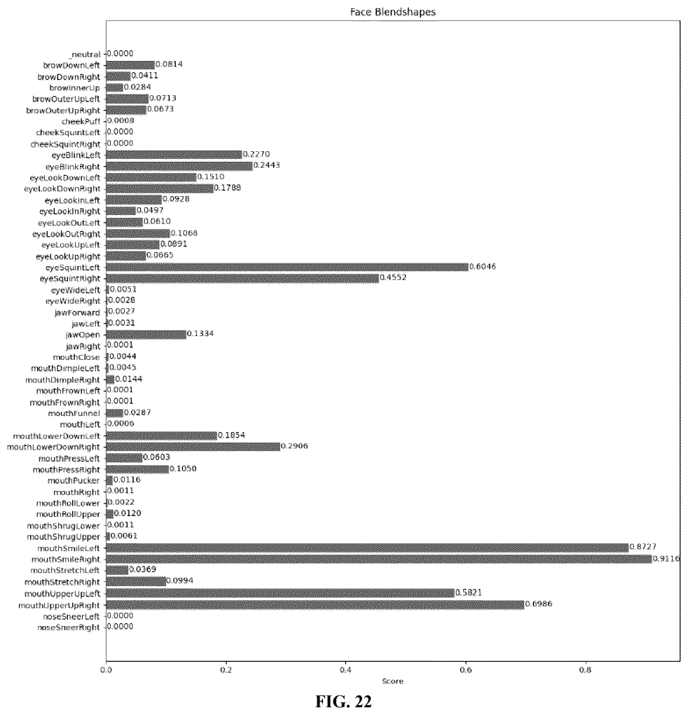

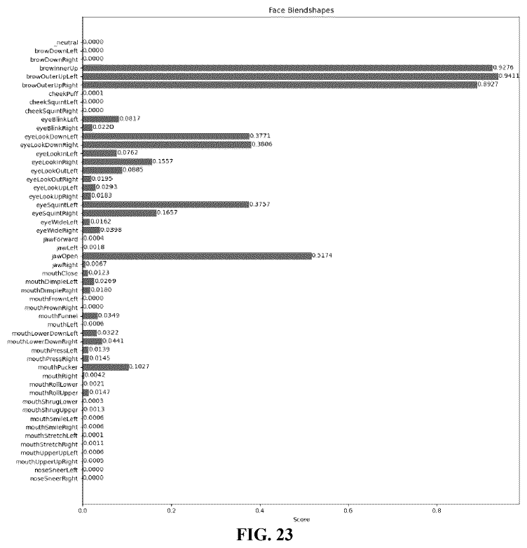

FIG. 23 is a graph with data from a specific subject, surprised-Subject #53.

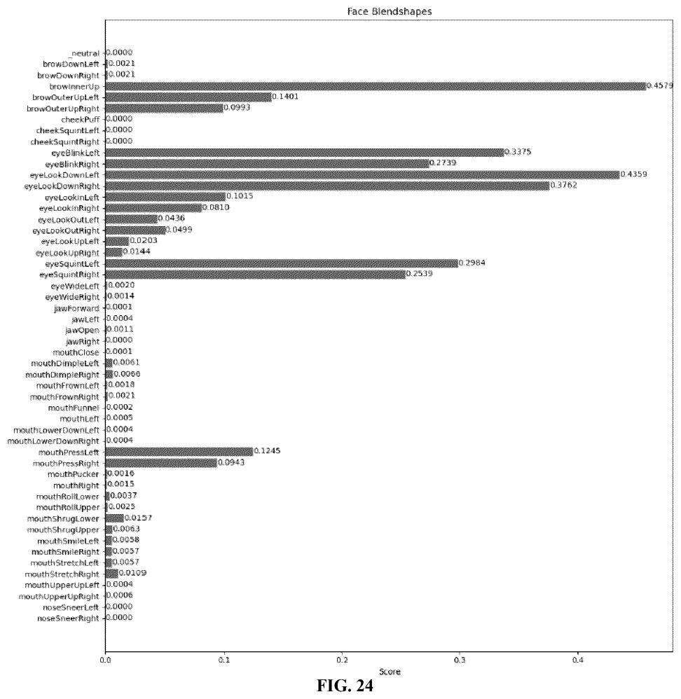

FIG. 24 is a graph with data from a specific subject, neutral-Subject #53.

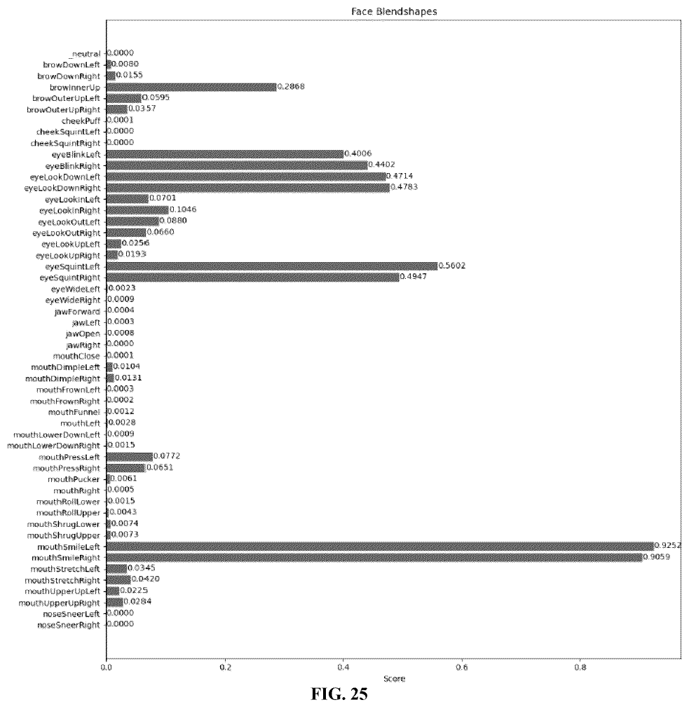

FIG. 25 is a graph with data from a specific subject, smile-Subject #53.

FIG. 26 is a graph with data from a specific subject, stressed-Subject #53.

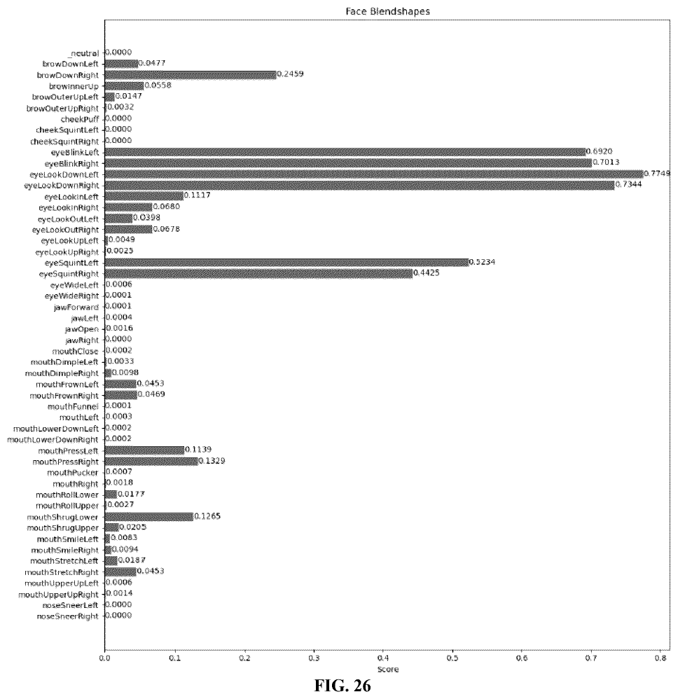

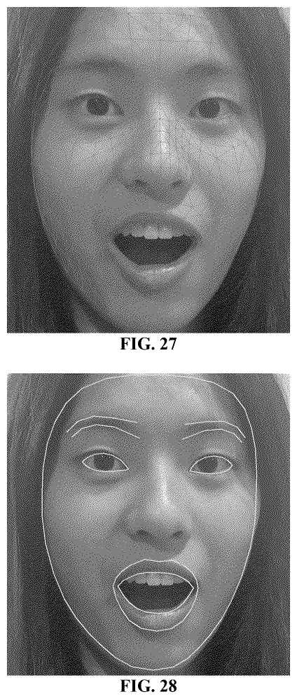

FIG. 27 shows an example of forming face tessellation. An example of code for calculating the tessellations is:

| | |
|---|---|
| Draw | face_mesh.FACEMESH_TESSELATION |

```
solutions.drawing_utils.draw_landmarks(
      image=annotated_image,
  landmark_list=face_landmarks_proto,
      connections=mp.solutions.face_mesh.FACEMESH_TESSELATION,
```

FIG. 28 shows an example of detection of the face contours. An example of code for detecting face contours is:

```
  Draw face_mesh.FACEMESH_CONTOURS solutions.drawing_utils.draw_landmarks(
    image=annotated_image,
landmark_list=face_landmarks_proto,
    connections=mp.solutions.face_mesh.FACEMESH_CONTOURS,
landmark_drawing_spec=None,
#connection_drawing_spec=mp.solutions.drawing_styles

[symbol]

[symbol]

indicates data missing or illegible when filed
```

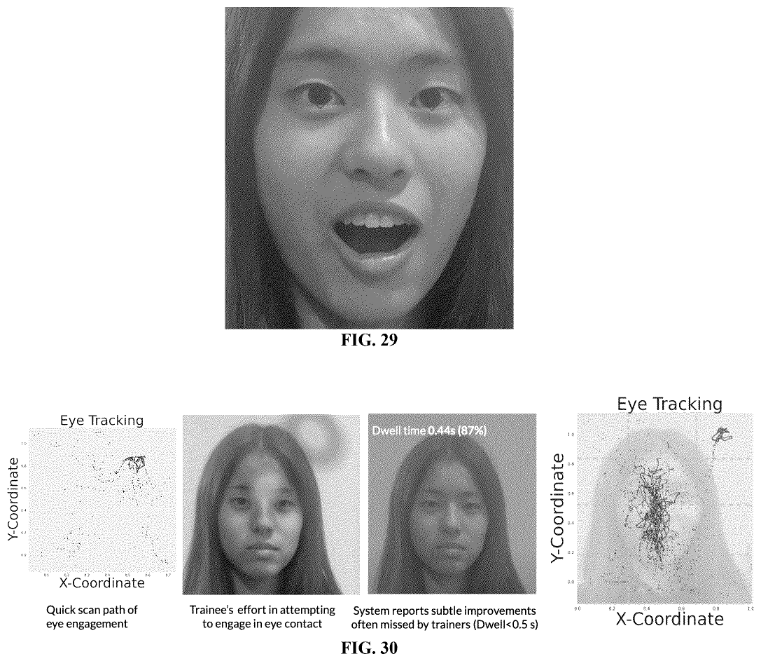

FIG. 29 shows an example of detection of the irises. An example of code for detecting the irises is:

| Draw FACEMESH_IRISES | |
|---|---|
| solutions.drawing_utils.draw_landmarks( image=annotate | |
| d_image, | landmark_list=face_landmarks_proto, |

```
  connections=mp.solutions.face_mesh.FACEMESH_IRISES,
  landmark_drawing_spec=None,
  connection_drawing_spec=DrawingSpecIris( ))
```

FIG. 30 compares results for eye tracking using the present invention comparing the dwell time.

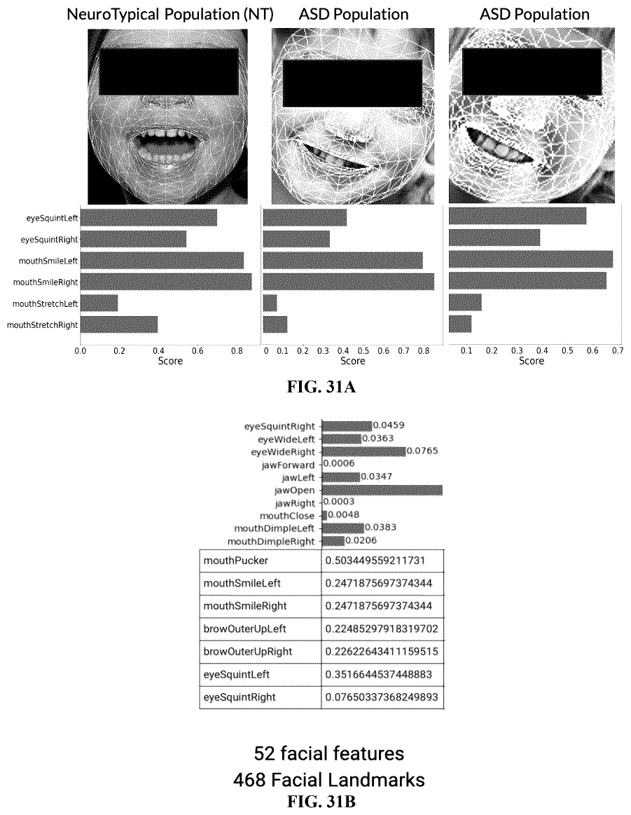

FIG. 31A shows the decomposition of facial emotions for 13 selected facial features.

FIG. 31B is a graph and chart that shows the results for the 13 essential facial features selected that are highly relevant to emotion expression.

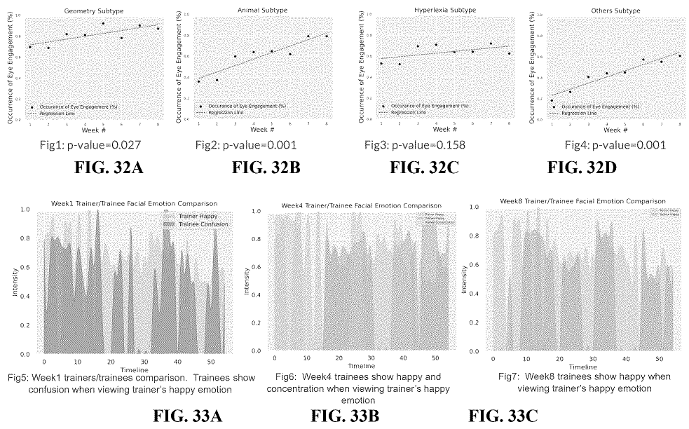

FIGS. 32A to 32D are graphs that show the reciprocal eye engagement: 3 out of 4 participant subtypes demonstrated statistically significant improvement with p-values 0.027, 0.001, 0.158, and 0.001 respectively.

FIGS. 33A to 33C are graphs that show the facial emotion response over time (weeks 1, week 4, and week 8). All 4 subtypes showed statistically significant improvement with digital twin.

A person of skill in the art would readily recognize that steps of various above-described methods can be performed by programmed computers. Herein, some embodiments are also intended to cover program storage devices, e.g., digital data storage media, which are machine or computer-readable and encode machine-executable or computer-executable programs of instructions, wherein said instructions perform some or all of the steps of said above-described methods. The program storage devices may be, e.g., digital memories, magnetic storage media such as magnetic disks and magnetic tapes, hard drives, or optically readable digital data storage media. The embodiments are also intended to cover computers programmed to perform said steps of the above-described methods.

A risk score of the present invention may be calculated with a machine learning algorithm using well-known statistical analysis techniques. Non-limiting examples of statistical analysis techniques that may be used to calculate the risk score include cross-correlation, Principal Components Analysis (PCA), factor rotation, Logistic Regression (Log Reg), Linear Discriminant Analysis (LDA), Eigengene Linear Discriminant Analysis (ELDA), Support Vector Machines (SVM), Random Forest (RF), Recursive Partitioning Tree (RPART), related decision tree classification techniques, Shrunken Centroids (SC), StepAIC, Kth-Nearest Neighbor, Boosting, Decision Trees, Neural Networks, Bayesian Networks, Support Vector Machines, and Hidden Markov Models, Linear Regression or classification algorithms, Nonlinear Regression or classification algorithms, analysis of variants (ANOVA), hierarchical analysis or clustering algorithms; hierarchical algorithms using decision trees; kernel based machine algorithms such as kernel partial least squares algorithms, kernel matching pursuit algorithms, kernel Fisher's discriminate analysis algorithms, or kernel principal components analysis algorithms. In an exemplary embodiment, the risk score is calculated as described in the examples.

The functions of the various elements shown in the figures, including any functional blocks labeled as “modules”, may be provided through the use of dedicated hardware as well as hardware capable of executing software in association with the appropriate software. When provided by a processor, the functions may be provided by a single dedicated processor, by a single shared processor, or by a plurality of individual processors, some of which may be shared. Moreover, explicit use of the term “module” should not be construed to refer exclusively to hardware capable of executing software, and may implicitly include, without limitation, digital signal processor (DSP) hardware, network processor, application-specific integrated circuit (ASIC), field-programmable gate array (FPGA), read-only memory (ROM) for storing software, random access memory (RAM), and nonvolatile storage. Other hardware, conventional and/or custom, may also be included.

It is contemplated that any embodiment discussed in this specification can be implemented with respect to any method or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

For each of the claims, each dependent claim can depend both from the independent claim and from each of the prior dependent claims for each and every claim so long as the prior claim provides a proper antecedent basis for a claim term or element.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit, and scope of the invention. All such similar substitutes and modifications apparent to those skilled in the art are deemed to be within the spirit, scope, and concept of the invention as defined by the appended claims.

### REFERENCES

[1] “Alexithymia, not autism, may drive eye-gaze patterns,” Spectrum | Autism Research News. Accessed: Dec. 29, 2023. [Online]. Available: www.spectrumnews.org/news/alexithymia-not-autism-may-drive-eye-gaze-patterns/
[2] X. Wang et al., “Prevalence and Correlates of Alexithymia and Its Relationship With Life Events in Chinese Adolescents With Depression During the COVID-19 Pandemic,” Front. Psychiatry, vol. 12, p. 774952, November 2021, doi: 10.3389/fpsyt.2021.774952.
[3] V. Gray, K. M. Douglas, and R. J. Porter, “Emotion processing in depression and anxiety disorders in older adults: systematic review,” BJPsych Open, vol. 7, no. 1, p. e7, December 2020, doi: 10.1192/bjo.2020.143.
[4] Y. Yi et al., “The percentage and clinical correlates of alexithymia in stable patients with schizophrenia,” Eur. Arch. Psychiatry Clin. Neurosci., vol. 273, no. 3, pp. 679-686, April 2023, doi: 10.1007/s00406-022-01492-8.
[5] P. E. Sifneos, R. Apfel-Savitz, and F. H. Frankel, “The phenomenon of ‘alexithymia’. Observations in neurotic and psychosomatic patients,” Psychother. Psychosom., vol. 28, no. 1-4, pp. 47-57, 1977, doi: 10.1159/000287043.
[6] “Toronto Alexithymia Scale (TAS-20) | Association for Contextual Behavioral Science.” Accessed: Dec. 29, 2023. [Online]. Available: https://contextualscience.org/TAS_Measure
[8] L. Calhoun, “Science Reveals the Right Timing for Eye Contact,” Inc.com. Accessed: Dec. 29, 2023. [Online]. Available: www.inc.com/lisa-calhoun/how-long-you-should-make-eye-contact-according-to-science.html
[9] “Why People With Autism Have Trouble Making Eye Contact,” Psychiatrist.com. Accessed: Dec. 29, 2023. [Online]. Available: www.psychiatrist.com/news/why-people-with-autism-have-trouble-making-eye-contact/
[10] C. Jongerius, R. S. Hessels, J. A. Romijn, E. M. A. Smets, and M. A. Hillen, “The Measurement of Eye Contact in Human Interactions: A Scoping Review,” J. Nonverbal Behav., vol. 44, no. 3, pp. 363-389, September 2020, doi: 10.1007/s10919-020-00333-3.
[11] I. F. Way et al., “Children's Alexithymia Measure (CAM): A New Instrument for Screening Difficulties with Emotional Expression,” J. Child Adolesc. Trauma, vol. 3, no. 4, pp. 303-318, November 2010, doi: 10.1080/19361521.2010.523778.
[12] A. Rosebrock, “Facial landmarks with dlib, OpenCV, and Python,” PyImageSearch. Accessed: Dec. 30, 2023. [Online]. Available: pyimagesearch.com/2017/04/03/facial-landmarks-dlib-opencv-python/
[14] N. Tottenham, M. E. Hertzig, K. Gillespie-Lynch, T. Gilhooly, A. J. Millner, and B. J. Casey, “Elevated amygdala response to faces and gaze aversion in autism spectrum disorder,” Soc. Cogn. Affect. Neurosci., vol. 9, no. 1, pp. 106-117, January 2014, doi: 10.1093/scan/nst050.
[15] “Autistics & eye contact (it's asynchronous) | Embrace Autism.” Accessed: Dec. 29, 2023. [Online]. Available: embrace-autism.com/autistics-and-eye-contact-its-asynchronous/
[16] S. Wohltjen and T. Wheatley, “Eye contact marks the rise and fall of shared attention in conversation,” Proc. Natl. Acad. Sci., vol. 118, no. 37, p. e2106645118, September 2021, doi: 10.1073/pnas.2106645118.

What is claimed is:

1. A method for training a subject diagnosed with a socio-emotional skills deficit using engagement training to enhance dyadic behavior, comprising:
selecting a first visual cue that will engage a visual attention of the subject;
exposing the subject to the first visual cue for one or more visual cue cycles comprising (1) a first time period with exposure of the subject to the visual cue, (2) a second time period that is a resting period in which the subject is not exposed to the first visual cue or the subject closes their eyes, and (3) a third time period in which the subject is not exposed to visual cues;
recording at least one of: a focus location on screen, one or more timestamps, tracking one or more directions of gaze, one or more blinks, or a reciprocal gaze with the first visual cue for one or more eyes of the subject in conjunction with exposure to the one or more visual cue cycles;
processing the recorded tracking one or more directions of gaze of the one or more eyes to determine a dwell time of the one or more directions of gaze toward the visual cue during the first time period of the one or more visual cue cycles; and
administering a treatment based on the recorded tracking by repeating the step of exposing the subject to the one or more visual cue cycles one or more times a day, until the dwell time of the eyes, reciprocal gaze, or both, exceeds a pre-set period of time,
wherein an increase of the dwell time, reciprocal gaze, or both, are indicative that the subject diagnosed with the socio-emotional skills deficit has increased focus and attention.

2. The method of claim 1, further comprising, once the dwell time, reciprocal gaze, or both of the eye meets or exceeds a preset period of time, then:
selecting a second visual cue that will engage the visual attention of the subject; and
repeating the steps of exposing, recording, and determining the gaze tracking of the subject to the second visual cue until the dwell time of the eyes exceeds a second pre-set period of time.

3. The method of claim 1, wherein the first visual cue is a trainer or video of a trainer on computer screen.

4. The method of claim 1, wherein the first time period is selected from 1, 1.5, 2, 2.5, 3, 3.5, 4, 4.5, 5, 5.5, 6, 6.5, 7, 7.5, 8, 8.5, 9, 9.5, 10, 15, 20, 25, or 30 seconds.

5. The method of claim 1, wherein the second time period is selected from 1, 2, 3, 4, 5, 6, 7, 8, 9, 10, 15, 20, 25, 30, 45, or 60 seconds.

6. The method of claim 1, wherein the third time period is selected from 1, 2, 3, 4, 5, 6, 7, 8, 9, 10, 15, 20, 25, 30, 45, 60 minutes, 2, 3, 4, 5, 6, 7, 8, 9, 10, 12, 14, 16, 18, 20, 24 hours, 2, 3, 4, 5, 6, 7 days.

7. The method of claim 1, wherein the first visual cue is selected from a face contour with a region covering the eyes, a cartoon of a face, a cartoon of an animal face, one or more neutral faces with one or more different age ranges, one or more different ethnicities, the face of the subject or a family member of the subject; or a trainer.

8. The method of claim 1, wherein the first visual cue can be one or more images, pre-recorded videos and live video feed from webcam, attached phone, or camera.

9. The method of claim 1, wherein a processor is programmed with a machine learning algorithm that calculates the position of one or more landmarks on a face selected from a position of the eye(s), eye contour, eyelid, pupil, a focus location on screen, one or more directions of gaze, blinks, dwell time, reciprocal eye engagement with a target in a training visual cue on a computer screen in conjunction with one or more timestamps during the exposure to the one or more visuals cue for one or more eyes of a subject; and
a. wherein the one or more directions of gaze are selected from: (1) right, left, center; (2) up or down; or (3) combinations of (1) and (2);
b. wherein the one or more landmarks on the face is calculated using 1, 2, 3, 4, 5, 6, 7, 8, 9, 10, 15, 20, 25, 30, 45, 60, 68, 70, 75, 80, 90, 100, 200, 300, 400, 500, 900, or 1000 facial landmarks.

10. The method of claim 1, wherein the one or more visual cue are repeated for 1, 2, 4, 5, 6, 7 days, 1, 2, 3, 4, 5, 6, 7, 8, 9, 10, 11, or 12 weeks, or 1, 2, 3, 4, 5, 6, 7, 8, 9, 10, 11, or 12 months until the dwell time, the reciprocal gaze, or both, meets or exceeds the pre-set period of time.

11. The method of claim 1, wherein the pre-set period of time is 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5 seconds.

12. The method of claim 1, wherein the first, the second and one or more additional visual cue(s) is/are, in order: (1) a cartoon of a face or a cartoon of an animal face; (2) one or more neutral faces with one or more age ranges or ethnicities; (3) one or more neutral faces with one or more different age ranges or ethnicities; (4) the face of the subject or a family member of the subject, and (5) a trainer.

13. The method of claim 1, further comprising using a machine learning algorithm to continuously modify: a length of exposure to one or more visual cues, to increase the dwell time, reciprocal gaze time, or both, of the one or more eyes of the subject on the visual cue.

14. The method of claim 1, wherein the alexithymia is related to the subject having at least one of: autism, neurotypical, depression, anxiety, schizophrenia, or a deficit in recognizing or describing emotions.

15. The method of claim 1, wherein the training is provided on a computer, laptop, tablet, phone, or other electronic or handheld device.

16. The method of claim 1, wherein the socio-emotional skills deficit has at least one of:
alexithymia, autism, depression, anxiety, or schizophrenia.

17. The method of claim 1, wherein the method is a self-paced multi-level training based on personal preferences (geometry preference, animal preference, hyperlexia).

18. The method of claim 1, wherein the method uses digital twin training partners to represent self, family members and therapists to at least one of: (1) reduce stimuli, (2) bridge a gap between self-intention and others' perception, and (3) practice the mirror neuron system.

19. The method of claim 1, wherein the method further comprises building a unique multimodal dataset to represent neurodivergent individuals' facial features, 3D tessellations and facial features; computer vision-based AI co-pilot feedback to augment neurological dysfunction; and optinally describing facial emotion expressions in measurable terms using selected facial features, such as mouthSmileLeft, mouthSmileRight, mouthUpperUpLeft, mouthUpperUpRight, browInnerUp, eyeSquintLeft, eyeSquintRight.

20. A method for facial emotional analysis of a subject, comprising:
recording or obtaining one or more images or video of a face of the subject;
detecting one or more facial feature in the one or more images or video of the face of the subject at least one of: a contour of the face, a focus location on a screen of one or more eyes, the irises of the one or more eyes, tracking one or more directions of gaze of the one or more eyes, a position of the one or more eyes, one or more blinks, a reciprocal gaze with a first visual cue for one or more eyes, a position of the mouth, a shape of the mouth, one or more eyebrows, a position of the one or more eyebrows, a share of the one or more eyebrows, of the subject in conjunction with exposure to the one or more visual cue cycles;
selecting a first visual cue that will engage a visual attention of the subject;
using a machine learning algorithm to detect one or more emotions selected from one or more selected from happy, sad, calm, triumph, aesthetic appreciations, relied, pride, admiration, adoration, contentment, satisfaction, love, excitement, interest, awe, amusement, joy, or ecstasy, in the facial features to generate a facial emotion analysis.

21. A device for training a subject diagnosed a socio-emotional skills deficit using engagement training to enhance dyadic behavior, comprising:
a memory;
a control circuitry functionally coupled to the memory, configured to:
deliver to the subject a first visual cue that will engage a visual attention of the subject for one or more visual cue cycles comprising:
(1) a first time period with exposure of the subject to the visual cue;
(2) a second time period that is a resting period in which the subject is not exposed to the first visual cue or the subject closes their eyes; and
(3) a third time period in which the subject is not exposed to visual cues;
one or more cameras capable or capturing an image/video of the subject in conjunction with one or more timestamps during the one or more visual cue cycles;
a processor that is programmed with a machine learning algorithm that processes the one or more captured image/video for the subject to calculate a position of one or more facial landmarks selected from at least one of: a position of the eyes, eye contour, eyelid, pupil, a focus location on a screen, one or more directions of gaze, one or more blinks, dwell time, or reciprocal eye engagement with target eyes in a training visual cue on a computer screen in conjunction with the one or more timestamps during exposure to the first visual cue for one or more eyes of the subject during the first time period of the one or more visual cue cycles; and
administering a treatment based on the recorded tracking by in which the subject is exposed to the one or more visual cue cycles one or more times a day, until the dwell time of the eyes, reciprocal gaze, or both, exceeds a pre-set period of time, and
wherein an increase of the dwell time, reciprocal gaze, or both, is indicative that the subject with the socio-emotional skills deficit has increased focus and attention.

22. The device of claim 18, further comprising a user interface, wherein the user interface is used to select from visual cues stored in said memory.

23. The device of claim 18, wherein the control circuitry is configured to determine when the dwell time, reciprocal gaze, or both, of the subject meets or exceeds the pre-set period of time.

24. The device of claim 18, wherein a second visual cue that will engage the visual attention of the subject and repeating the steps of exposing, recording, and determining a period of the gaze of the subject to the second visual cue until the dwell time, reciprocal gaze, or both, of the eyes exceeds a second pre-set period of time.

25. The device of claim 18, wherein the first time period is selected from 1, 1.5, 2, 2.5, 3, 3.5, 4, 4.5, 5, 5.5, 6, 6.5, 7, 7.5, 8, 8.5, 9, 9.5, 10, 15, 20, 25, or 30 seconds.

26. The device of claim 18, wherein the second time period is selected from 1, 2, 3, 4, 5, 6, 7, 8, 9, 10, 15, 20, 25, 30, 45, or 60 seconds.

27. The device of claim 18, wherein the third time period is selected from 1, 2, 3, 4, 5, 6, 7, 8, 9, 10, 15, 20, 25, 30, 45, 60, minutes, 2, 3, 4, 5, 6, 7, 8, 9, 10, 12, 14, 16, 18, 20, 24 hours, 2, 3, 4, 5, 6, 7 days.

28. The device of claim 18, wherein the first visual cue is selected from a face contour with a region covering the eyes, a cartoon of a face, a cartoon of an animal face, one or more neutral faces with one or more different age ranges, one or more different ethnicities, the face of the subject or a family member of the subject, or a trainer.

29. The device of claim 18, wherein the visual cue is selected from at least one of: one or more images, one or more pre-recorded videos, one or more live video feeds, from a webcam, a phone, or a camera.

30. The device of claim 18, wherein a processor is programmed with a machine learning algorithm that calculates the position of one or more landmarks on a face selected from a position of the eye(s), eye contour, eyelid, pupil, a focus location on screen, one or more directions of gaze, blinks, dwell time, reciprocal eye engagement with a target in a training visual cue on a computer screen in conjunction with one or more timestamps during the exposure to the one or more visuals cue for one 25 or more eyes of a subject; and
a. wherein the one or more directions of gaze are selected from: (1) right, left, center; (2) up or down; or (3) combinations of (1) and (2);
b. wherein the one or more landmarks on the face is calculated using 1, 2, 3, 4, 5, 6, 7, 8, 9, 10, 15, 20, 25, 30, 45, 60, 68, 70, 75, 80, 90, 100, 200, 300, 400, 500, 900, or 1000 facial landmarks.

31. The device of claim 18, wherein the one or more visual cue cycles-are repeated for 1, 2, 4, 5, 6, 7 days, 1, 2, 3, 4, 5, 6, 7, 8, 9, 10, 11, or 12 weeks, or 1, 2, 3, 4, 5, 6, 7, 8, 9, 10, 11, or 12 months until the dwell time, the reciprocal gaze, or both, meets or exceeds the pre-set period of time.

32. The device of claim 18, wherein the pre-set period of time is 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, or 3.0, 3.1, 3.2, 3.3, 3.4, 3.5 seconds.

33. The device of claim 18, wherein the first, the second, and one or more additional visual cue(s) is/are, in order: (1) a cartoon of a face or a cartoon of an animal face; (2) one or more neutral faces with one or more different age ranges, (3) one or more different ethnicities; (4) the face of the subject or a family member of the subject, and (5) a trainer.

34. The device of claim 18, further comprising programming the processor with a machine learning algorithm to continuously modify: a length of exposure to one or more visual cues, to increase the dwell time, reciprocal gaze time, or both, of the eyes of the subject on the visual cue.

35. The device of claim 18, wherein a focus of the position of the eyes on a device screen is computed using a machine learning algorithm based on one or more facial landmarks of the subject and real time changes when tracking a predefined moving visual cue on a device screen.

36. The device of claim 18, further comprising displaying at least one of: one or more dwell times during and between one or more visual cue cycles; average dwell times across one or more visual cue cycles; average dwell times over various days, weeks, or months; or
for a new visual cue one or more dwell times during and between one or more visual cue cycles; average dwell times across one or more visual cue cycles; average dwell times over various days, weeks, or months.

37. The device of claim 18, further comprising displaying at least one of: one or more reciprocal gaze times during and between one or more visual cue cycles; average reciprocal gaze times across one or more visual cue cycles; average reciprocal gaze times over various days, weeks, or months; or
for a subsequent visual cue one or more reciprocal gaze times during and between one or more visual cue cycles; average reciprocal gaze times across one or more visual cue cycles; average reciprocal gaze times over various days, weeks, or months.

38. The device of claim 18, wherein the alexithymia is related to the subject having one or multiple conditions selected from: autism, neurotypical, depression, anxiety, or schizophrenia or a subject with a deficit in recognizing or describing emotions.

39. The device of claim 18, wherein the training is provided on a computer, laptop, tablet, phone, or other electronic or handheld device.

40. The device of claim 18, wherein the socio-emotional skills deficit has at least one of: alexithymia, autism, depression, anxiety, or schizophrenia.

41. A device for generating a facial emotion analysis, comprising:
recording or obtaining one or more images or video of a face of the subject;
detecting one or more facial feature in the one or more images or video of the face of the subject at least one of: a contour of the face, a focus location on a screen of one or more eyes, the irises of the one or more eyes, tracking one or more directions of gaze of the one or more eyes, a position of the one or more eyes, one or more blinks, a reciprocal gaze with a first visual cue for one or more eyes, a position of the mouth, a shape of the mouth, one or more eyebrows, a position of the one or more eyebrows, a share of the one or more eyebrows, of the subject in conjunction with exposure to the one or more visual cue cycles;
selecting a first visual cue that will engage a visual attention of the subject;
using a machine learning algorithm to detect one or more emotions selected from triumph, aesthetic appreciations, relied, pride, admiration, adoration, contentment, satisfaction, love, excitement, interest, awe, amusement, joy, or ecstasy, in the facial features to generate a facial emotion analysis.

42. A computer or electronic system, comprising:
one or more processors; and
one or more hardware storage devices having stored thereon computer-executable instructions that, when executed by the one or more processors, configure the computer system to perform at least the following:
selecting a first visual cue that will engage a visual attention of the subject;
exposing the subject to the visual cue for one or more visual cue cycles comprising (1) a first time period with exposure of the subject to the visual cue, (2) a second time period that is a resting period in which the subject is not exposed to the first visual cue or the subject closes their eyes, and (3) a third time period in which the subject is not exposed to visual cues;
recording a focus location on a screen, one or more timestamps, one or more directions of gaze, one or more blinks, or a reciprocal gaze with a trainer or a video of a trainer on computer screen for the one or more eyes of the subject to track a gaze in conjunction with exposure to the visual cue;
processing the recorded gaze tracking of the one or more eyes to determine a dwell time of the gaze toward the visual cue during the first time period of the one or more visual cue cycles; and
administering a treatment based on the recorded tracking by repeating the step of exposing the subject to the one or more visual cue cycles one or more times a day, until the dwell time of the eyes exceeds a pre-set period of time,
wherein an increase of the dwell time, reciprocal gaze, or both, are indicative that the subject diagnosed with alexithymia or a subject with deficit in socio-emotional skills has increased focus and attention.

43. The computer or electronic system of claim 39, further comprising, once the dwell time, reciprocal gaze, or both, of the eye meets or exceeds the pre-set period of time, then:
selecting a second visual cue that will engage the visual attention of the subject; and
repeating the steps of exposing, recording, and determining a period of the gaze of the subject to the second visual cue until the dwell time of the eyes exceeds a second pre-set period of time.

44. The computer or electronic system of claim 39, wherein the first time period is selected from 1, 1.5, 2, 2.5, 3, 3.5, 4, 4.5, 5, 5.5, 6, 6.5, 7, 7.5, 8, 8.5, 9, 9.5, 10, 15, 20, 25, or 30 seconds.

45. The computer or electronic system of claim 39, wherein the second time period is selected from 1, 2, 3, 4, 5, 6, 7, 8, 9, 10, 15, 20, 25, 30, 45, or 60 seconds.

46. The computer or electronic system of claim 39, wherein the third time period is selected from 1, 2, 3, 4, 5, 6, 7, 8, 9, 10, 15, 20, 25, 30, 45, 60, minutes, 2, 3, 4, 5, 6, 7, 8, 9, 10, 12, 14, 16, 18, 20, 24 hours, 2, 3, 4, 5, 6, 7 days.

47. The computer or electronic system of claim 39, wherein the first visual cue is selected from a face contour with a region covering the eyes; a cartoon of a face, a cartoon of an animal face, one or more neutral faces with one or more different age ranges or ethnicities; the face of the subject or a family member of the subject; or a trainer.

48. The computer or electronic system of claim 39, wherein the first visual cue can be one or more images, pre-recorded videos and live video feed from webcam, attached phone, or camera.

49. The computer or electronic system of claim 39, wherein the processor is programmed with a machine learning algorithm that calculates the landmarks on the face including but not limited to the position of the eyes (eye contour, eyelid, pupil), the focus location on screen, directions of gaze, blinks, dwell time, and/or reciprocal eye engagement with the target's eyes in the training visual cue on computer screen in conjunction with the timestamps of the exposure to the visual cue for one or more eyes of a subject.

50. The computer or electronic system of claim 39, wherein the one or more visual cue are repeated for 1, 2, 4, 5, 6, 7 days, 1, 2, 3, 4, 5, 6, 7, 8, 9, 10, 11, or 12 weeks, or 1, 2, 3, 4, 5, 6, 7, 8, 9, 10, 11, or 12 months until the dwell time, reciprocal gaze, or both, meets or exceeds the pre-set period of time.

51. The computer or electronic system of claim 32, wherein the pre-set period of time is 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, or 2.9, 3.0, 3.1, 3.2, 3.3, 3.4, 3.5 seconds.

52. The computer or electronic system of claim 39, wherein the first, second and additional visual cue(s) is/are, in order: (1) a cartoon of a face or a cartoon of an animal face; (2) one or more neutral faces with one or more different age ranges or ethnicities; (3) the face of the subject or a family member of the subject, and (4) a trainer.

53. The computer or electronic system of claim 39, further comprising using a machine learning algorithm to continuously modify: a length of exposure to one or more visual cues, to increase the dwell time, reciprocal gaze, or both, of the eyes of the subject on the visual cue.

54. The computer or electronic system of claim 39, wherein the alexithymia is related to the subject having one or more conditions selected from: autism, neurotypical, depression, anxiety, or schizophrenia or a subject with a deficit in recognizing or describing emotions.

55. The computer or electronic system of claim 39, wherein the training is provided on a computer, laptop, tablet, phone, or other electronic or handheld device.

56. A computer or electronic system for determining a facial emotional analysis, comprising:
recording or obtaining one or more images or video of a face of the subject;
detecting one or more facial feature in the one or more images or video of the face of the subject at least one of: a contour of the face, a focus location on a screen of one or more eyes, the irises of the one or more eyes, tracking one or more directions of gaze of the one or more eyes, a position of the one or more eyes, one or more blinks, a reciprocal gaze with a first visual cue for one or more eyes, a position of the mouth, a shape of the mouth, one or more eyebrows, a position of the one or more eyebrows, a share of the one or more eyebrows, of the subject in conjunction with exposure to the one or more visual cue cycles;
selecting a first visual cue that will engage a visual attention of the subject;
using a machine learning algorithm to detect one or more emotions selected from triumph, aesthetic appreciations, relied, pride, admiration, adoration, contentment, satisfaction, love, excitement, interest, awe, amusement, joy, or ecstasy, in the facial features to generate a facial emotion analysis.
